(19) United States
(12) Patent Application Publication
(10) Pub. No.: US 2025/0346917 A1
(43) Pub. Date: Nov. 13, 2025

(54) METHODS FOR THE SELECTION OF NUCLEIC ACID SEQUENCES

(71) Applicant: Cytiva Sweden AB, Uppsala (SE)

(72) Inventors: Andreas Jonsson, Niskayuna, NY (US); Daniel Ivansson, Uppsala (SE)

(21) Appl. No.: 17/995,478

(22) PCT Filed: Apr. 6, 2021
(86) PCT No.: PCT/EP2021/058918

§ 371 Date: Oct. 4, 2022

(30) Foreign Application Priority Data
Apr. 8, 2020 (GB) 2005179.3

(51) Int. Cl. C12N 15/85; C12N 15/62; C12Q 1/6869; G01N 33/58

(52) U.S. Cl. CPC C12N 15/85; C12N 15/62; C12Q 1/6869; G01N 33/582

(57) ABSTRACT

The present disclosure relates to a method for targeted integration of a donor vector into a specific pre-defined genomic location of an isolated eukaryotic host cell. The vector and host cell together comprise nucleic acid components allowing for the selection of cells having integrated the donor vector into the pre-defined genomic location of the host cell. More specifically, the present method provides for the selection of sequence optimized nucleic acid sequence variants. Such optimized nucleic acid sequence variants may comprise sequence optimized expression vector components for subsequent use in recombinant protein production.

### TECHNICAL FIELD OF THE INVENTION

The present invention relates to the field of cell-based methods utilizing targeted integration of a donor vector into a specific pre-defined genomic location of a eukaryotic host cell genome, wherein said vector and host cell comprises nucleic acid components rendering it possible to selectively choose those cells having integrated the donor vector into a pre-defined genomic location of the host cell genome and optionally to detect and remove cells having undergone any additional random integration events into other parts of the genome. The present invention is particularly directed to the use of such targeted integration systems for evaluating libraries of nucleic acid sequence variants with the aim to identify optimized nucleic acid sequence variants therefrom. Such optimized nucleic acid sequence variants can subsequently be used in expression systems for recombinant protein production or in other biotechnology applications.

### BACKGROUND OF THE INVENTION

Optimizing expression cassettes for commercial production of proteins During the last 30 years recombinant protein therapeutics has evolved from a novelty to a dominating position among marketed drugs. Recombinant production of therapeutic proteins has surpassed the 100 billion $ per year market volume and plays an important role in the global economy as well as in advanced medical care. The therapeutic protein class includes replacement proteins (insulin, growth factors, cytokines and blood factors), vaccines (antigens, VLPs) and monoclonal antibodies. The by far dominating format is the monoclonal antibodies [1, 2]. Through the continued advancement of protein engineering and synthetic biology, the therapeutic protein class is becoming highly diversified with rapid growth in the development of engineered protein formats such as bi- and multi-specific antibodies [3, 4]. Some of the recombinant proteins can be produced in simple microbial cells such as E. coli, but for more complex proteins, including both the traditional monoclonal antibody class and the emerging engineered antibody class, Chinese Hamster Ovary (CHO) cells is the dominating host for production [2].

The dominating approach to generate a high performance therapeutic protein producing cell line within the industry today is to introduce the recombinant protein genes into the genome of a host CHO cell line via a random integration approach and select/screen for individual cells having integrated the genes at active genomic sites at a copy number yielding sufficiently high transcription and that at the same time having a phenotype capable of supporting high protein translation and secretion. This is a highly work intensive and time-consuming process with large inherent uncertainties and biological variation. Typical process duration spans between 3-12 months depending on the growth of the host cells, the level of automation implemented and the end point (for example if assessment of long-term clone stability is included).

One fundamental limitation associated with the random integration approach is the low sampling of the cellular diversity in a transfected pool of cells. Only around 0.1-1% of the transfected cells integrate recombinant DNA. Further, this sub-population is highly heterogeneous in terms of integration locations, copy number and integrity of the integrated DNA. Adding the inherent global phenotypic variation of CHO cells, which is inherent for CHO cells due to the high genomic and epi-genomic plasticity, makes finding a high producing clone like finding a needle in a haystack. This also explains why a high variation in protein production from non-clonal stable pools is generally observed (stochastic sampling of phenotypic diversity).

This under-sampling and high biological noise also make comparison of different gene cassette designs of a therapeutic protein candidate for optimization of expression difficult. Comparison of multiple variants via parallel generation of stable pools is highly work intensive and the high biological noise will make results unreliable. Use of simultaneous transfection of variant libraries is hampered by the fact that random integration typically results in integration of multiple copies of an expression vector and hence any cell generated through such a workflow will typically contain integrated copies from more than one gene cassette design. Improving protein expression by Cell Line engineering strategies based on random integration of effector genes is hampered by the same reasons.

One potentially major improvement to all of the above limitations is to utilize targeted integration (Site-Directed Integration; SDI) of Genes of Interest (GOI's). In such a scenario a pre-identified genomic location known to support high and stable transcription is used as a target destination for GOI's. Using intelligent combinations of pre-introduced sequences and vector designs, including the use of co-transfected nucleic acid enzymes such as nucleases or recombinases, will facilitate targeted insertion and ensure that all cells in culture will contain correctly inserted GOIs and hence have a high transcription rate. This will significantly reduce the number of clones needed in a screening campaign for Cell Line Development (CLD) and reduce biological noise in comparisons of gene cassette designs or Cell Line engineering efforts. Multiple technical solutions for targeted integration are described in the art [5-8]. However, despite so, challenges remain.

The Flp-In™ system (based on the Flp recombinase, also referred to as Flippase recombinase) for targeted integration [9] is an example of a solution utilizing a single recombinase recognition sequence in combination with its recombinase to enable targeted integration at a pre-defined genomic location. Following the action of the recombinase the complete expression vector is integrated at the recombinase recognition sequence. Cells with correct integration events can be selected as integration at the recombinase recognition site inactivates one selection marker and activates a second selection marker. Major drawbacks with this solution are (i) there is no mechanism to detect or remove cells having integrated additional copies of the expression vector by random integration events, (ii) there is no mechanism to remove sequence regions, such as plasmid backbone sequences and active selection marker genes, that can be negative to the expression of the GOI, (iii) the method has questionable flexibility in the choice of selection marker as activation of the selection marker during integration results in the fusion of extra amino acids at the N-terminal that can impact it's functionality and (iv) selection is based on antibiotic resistance which requires prolonged time and introduces potential biases based on differential growth rates among cells with positive integration.

To avoid presence of sequences with a potential negative impact on GOI expression following targeted integration, different solutions for cassette exchange reactions at a pre-defined genomic location has been described in the art [5-8]. An example of such a solution has been disclosed by Rentschler [10]. The pre-defined genomic location utilizes an active selection marker gene (GFP) flanked by two orthogonal recombinase recognition sequences both targets for the same recombinase. The GOI in the expression vector is in turn flanked by two recombinase recognition sequences matching the two present in the genome. Upon action of the recombinase, cassette exchange between the selection marker cassette and the GOI cassette can occur. Cells having undergone the cassette exchange can be selected by absence of GFP expression. Drawbacks for this kind of solution are (i) there is no mechanism to detect or remove cells having integrated additional copies of the expression vector by random integration events, (ii) as selection of cells having undergone cassette exchange is based on absence of an initially active gene product, the time point for selection must be delayed to allow for degradation/dilution of GFP. Besides prolonging the workflow this also introduces potential biases based on differential growth rates among cells with positive integration, as mentioned above.

Haghighat-Khah R E, et al. discloses a two-step site-specific cassette exchange system in insects, i.e. the Aedes aegypti Mosquito and the Plutella xylostella moth [11]. The exchange system utilizes a phiC31 recombinase for integration of an expression vector at a pre-defined genomic location followed by the use of a second recombinase (Cre or Flp) for excision of plasmid backbone sequences. However, the exchange system of Haghighat Khah R E, et al. does not provide means for distinguishing between targeted integration and random integration events. In addition, no means to remove the selection marker gene are provided.

Yuan, Y; et al. discloses a recombinase-based method to produce selection marker- and vector-backbone-free transgenic cells utilizing PhiC31-mediated gene delivery into pseudo-attP sequences present naturally in the genome of the targeted cells [12]. Selection of cells in which integration has occurred is achieved via presence of an active eGFP expression cassette in the expression vector and an att-B-TK fusion gene becoming inactivated upon targeted integration was used as a negative selection marker to eliminate random integration events in a second selection step. The selection system and the plasmid bacterial backbone was subsequently excised by using the two other recombinases Cre and Dre. Critical drawbacks in the method disclosed by Yuan, Y; et al. for adoption to recombinant protein production applications based on integration into one pre-defined genomic location are (i) the method does not provide means to distinguish between cells having undergone integration at the pre-defined location from cells having undergone integration at a random pseudo-attP site as inactive TK genes would result from both scenarios, (ii) the first selection step cannot be performed until transient expression of the selection marker has vanished which adds time and introduces potential biases based on differential growth rates among cells with positive integration, (iii) the first selection step does not distinguish between desired integration, integration at a pseudo-attP site or a random integration event.

Parthiban, K; et al. discloses a cassette-exchange method based on nuclease-directed integration of full-length IgG-formatted antibody genes into mammalian cells to create massive repertoires of cells enriched for cells encompassing one antibody gene per cell [13]. Selection of cells for which integration has occurred at the desired genomic location is based on activation of a blasticidin resistance gene by an endogenous promotor naturally present at the chosen genomic location. Drawbacks of this solution includes (i) there is no mechanism to detect or remove cells having integrated additional copies of the expression vector by random integration events and (ii) there is no mechanism to remove the selection marker cassette which can have a negative impact on expression of the GOI.

For applications based on integration of nucleic acid sequence libraries in particular, there are also a critical need to develop methods that can achieve higher integration efficiencies as this dictates the upper diversity of libraries that can be efficiently evaluated.

Accordingly, there is still a need in the art to identify improved expression systems for the production of recombinant proteins. In particular, to enable improved means to optimize the expression cassette of a therapeutic protein candidate, there is an urgent need for novel methods that can efficiently integrate a donor vector and enable fast and precise selection of the cells having integrated the insert into the correct position of the host cell genome and simultaneously enable removal of cells having integrated additional copies at random genomic locations.

### Nucleic Acid Sequence Optimization

Every protein has an upper expression potential ultimately dictated by its amino acid sequence and different sequences can result in large differences in expression potential. Hence, minor changes of the amino acid sequence of a therapeutic protein candidate outside its target interaction surface can be critical to improve expression levels. If amino acid changes cannot be introduced due to risk mitigation of clinical complications, promotors with different strengths can be needed to avoid overwhelming of the cellular machinery. In addition, many studies performed during the last 10-20 years (See [14-19] for a few) have shown that the expression levels for a given protein (fixed amino acid sequence) can vary greatly based on the use of different sequence components in the vector (5′-UTR sequence, Signal Peptide sequence, synonymous coding nucleotide sequences and 3′-UTR sequences) and that well performing combinations are at least partly protein dependent.

Traditionally, sequence components have been cloned from nature. However, there are good reasons to believe that natural sequence elements are not optimal for maximal expression of a single defined protein during bioprocess conditions. After all, they have evolved in the context of a whole organism with all the constraints that this implies. With the increasing diversity of therapeutic protein formats being explored and the rapid maturation of synthetic biology (enabling construction and evaluation of new sequence variants not present in nature and with bp precision) the sequence-based design space for protein expression is daunting.

Due to this, novel measures of identifying optimized sequence variants of nucleic acid sequences encoding or impacting proteins of interest are also continuously sought, such measures playing a significant role in the total expression potential of a protein of interest in a recombinant cell system.

Hence, there still exist a large untapped potential for improving commercial protein production or function of recombinant proteins by enabling assessment of a wider diversity of sequence variants in the final production system, both of vector components and of nucleic acid sequences encoding proteins of interest before selecting the final sequence combination for production.

### SUMMARY OF THE INVENTION

The above-mentioned problems have now been solved or at least mitigated by the provision of methods and means presented further herein.

The present disclosure provides a novel solution for recombinant protein production utilizing Site Directed Integration (SDI) of a single copy of a donor vector into a pre-defined genomic location of an isolated eukaryotic host cell. The SDI-based system of the present disclosure is based on a unique and inventive combination of well-established nucleic acid components for the efficient integration of a donor vector into a dedicated target site of the host cell. The method provides for the specific positive selection of host cells having integrated the donor vector into the dedicated pre-defined genomic location. The method also provides for, by negative selection, detecting and optionally removing any cells for which undesired integration events have occurred in other locations of the host cell genome. This two-step selection method is unique and will be very useful in the field of recombinant protein production and especially so for enabling efficient, non-biased evaluation of nucleic acid sequence variants with an impact on recombinant protein production or function.

As mentioned previously herein, a critical component for improved cell line development, flexible cell line engineering and enabling of advanced applications such as simultaneous probing of gene construct libraries, is increased control of recombinant gene integration into host cell lines and better control over their copy number. This is now provided by the present disclosure. Initially, Chinese Hamster Ovary (CHO) cells were used to set up a method presented herein as putative hot spot locations has been identified, but the SDI system should be applicable to any eukaryotic cell system including mammalian cells, such as human cells.

More specifically, the present disclosure provides a novel solution for optimization of nucleic acid sequences for use in recombinant protein production utilizing said Site Directed Integration (SDI) system capable of integrating a single copy of a donor vector into a pre-defined genomic location of an isolated eukaryotic host cell. The sequence optimization method comprises the generation of a library of nucleic acid sequence variants of donor vector components, such as promoters, IRES or enhancer sequences, or of nucleic acid sequences encoding proteins of interest, followed by targeted integration of said nucleic acid variants into a plurality of host cells, to identify an optimized nucleic acid sequence variant therefrom.

Accordingly, in a first aspect, the present invention relates to a method for the selection of a sequence optimized nucleic acid sequence from a plurality of nucleic acid sequence variants, wherein said sequence optimized nucleic acid sequence corresponds to a eukaryotic cell with a defined phenotype, said method comprising:
i) Providing a population of eukaryotic cells, each cell comprising a pre-defined genomic location, which pre-defined genomic location comprises:
a. a nucleic acid sequence I1 comprising a recognition site for a first DNA enzyme;
b. a nucleic acid sequence E1 comprising a recognition site for a second DNA enzyme; and
c. a promotor nucleic acid sequence;
ii) Providing a plurality of donor vectors, each donor vector comprising:
a. a nucleic acid sequence I2;
b. a nucleic acid sequence E2 comprising a recognition site for said second DNA enzyme;
c. a nucleic acid sequence encoding a first selection marker; and
d. a nucleic acid sequence region comprising a nucleic acid sequence variant,
iii) Contacting the plurality of donor vectors with the population of cells in the presence of a first DNA enzyme, wherein the presence of the first DNA enzyme enables recombination between the nucleic acid sequence I2 of a donor vector and the nucleic acid sequence I1 present in the pre-defined genomic location of a cell;
iv) Selecting and isolating a cell having a donor vector integrated at the pre-defined genomic location by detecting the expression of the first selection marker in the cell, wherein the expression of the first selection marker is activated by the promotor nucleic acid sequence at the pre-defined genomic location; and
v) Selecting and isolating a cell with a defined phenotype from the cells of step iv), thereby selecting and isolating a sequence optimized nucleic acid sequence from said nucleic acid sequence variants, said sequence optimized nucleic acid sequence corresponding to said defined phenotype.

In another aspect, there is provided herein a sequence optimized nucleic acid sequence selected by a method as disclosed herein.

In yet another aspect, there is provided the use of a sequence optimized nucleic acid sequence, for producing a recombinant protein.

In yet another aspect, there is provided an isolated eukaryotic cell with a defined phenotype corresponding to a sequence optimized nucleic acid sequence obtainable by the method as disclosed herein.

### BRIEF DESCRIPTION OF THE DRAWINGS

FIG. 1 shows a schematic illustration of the general concept of a method for targeted integration of a plurality of donor vectors into a pre-defined genomic location of a plurality of eukaryotic host cells via the use of at least two DNA enzymes with orthogonal specificity. The targeted integration of the plurality of donor vectors into the pre-defined location of a plurality of cells is followed by an isolation/enrichment of a defined phenotype and subsequent use thereof. SOI=Sequence of interest.

FIG. 2 shows a schematic illustration of the general concept of a method for targeted integration of a donor vector into a pre-defined genomic location of a host cell via the use of at least two DNA enzymes with orthogonal specificity.

FIG. 3 shows a schematic illustration of the Landing Pad designs (nucleic acid sequences present in the pre-defined genomic locations of the host cell genome) and matching Donor Vectors for the HyClone LP1P1 and HyClone LP2P2 cell lines.

FIG. 4 illustrates an example of Flow Cytometry plots at day 7 post transfection in comparison to a non-transfected control (NC). Density of cells in plots having a concentrated main population is visualized by alternating black and white regions (20% of total cells in each region). Upper row shows FACS data for a non-transfection control (NC) culture of HyClone CHO cells. Middle row shows FACS data from a random integration control (RI) based on HyClone CHO cells (lacking LP) transfected with the Donor Vector B only (without PhiC31). Lower row shows FACS data for the HyClone CHO LP2P2 cell line transfected with PhiC31 and Donor Vector B (SDI). The gate (B, D, F) in middle plot of each row is set based on the non-transfected control and reports percentage of cells having activated the selection marker above background.

FIG. 5 shows a schematic illustration of the Landing Pad Cell line and Donor Vector used as well as the alterations at the Landing Pad expected to occur through the activity of PhiC31 (1) and Cre (2).

FIG. 6 shows flow cytometry plots of SDI populations at day 7 post transfection of Cre recombinase variants in comparison to a negative mock transfection control. Density of cells in plots having a concentrated main population is visualized by alternating black and white regions (20% of total cells in each region). Upper panel shows plots for a mock transfection lacking Cre recombinase encoding nucleic acid molecules, middle panel shows plots of a population transfected with a Cre recombinase expression plasmid and the lower panel shows plots of a population transfected with synthetic Cre recombinase mRNA.

FIG. 7 shows a schematic illustration of the Landing Pad Cell line and Donor Vectors used as well as the alterations at the Landing Pad expected to occur through the activity of PhiC31 recombinase (1) and Cre recombinase (2).

FIG. 8 shows flow cytometry plots from the steps performed according to FIG. 7. Density of cells in plots having a concentrated main population is visualized by alternating black and white regions (20% of total cells in each region). The left plot in the upper panel show the population following the second eGFP (Green Fluorescent Protein) positive sort. The middle plot in the upper panel show the population 7 days post Cre recombinase transfection. The right plot in the upper panel show the population after the eGFP negative sort performed according to gate E following Cre recombinase transfection. The lower panel show plots of the population seven days post transfection of Step2 Sort cells with DNA Donor Vector B.

FIG. 9 shows a schematic illustration of the Landing Pad Cell line and Donor Vectors used. GSx=Glutamine Synthetase gene variant.

FIG. 10 shows flow cytometry plots from the generation of a cell population using SDI of the Donor vector. Density of cells in plots having a concentrated main population is visualized by alternating black and white regions (20% of total cells in each region). Upper panel shows a plot of a population having undergone G418 selection, RFP (Red Fluorescent Protein) positive FACS sorting and transfection with synthetic Cre recombinase mRNA. eGFP histograms are shown for both the RFP negative sub-population (corresponds to integration at the Landing Pad with Cre recombinase mediated excision of TagRFP-T) and the RFP positive sub-population (corresponds to failed Cre recombinase mediated excision of TagRFP-T that can be caused by off-target integration or truncated integration at the Landing Pad). The lower panel shows plots of the final SDI pool generated using a FACS sort of RFP negative/GFP positive cells from the upper panel.

FIG. 11 shows the first selection marker of said donor vector linked to a gene coding for said second DNA Enzyme via an IRES element. Both the first selection marker and the second DNA Enzyme are activated upon integration at the pre-defined genomic location.

FIG. 12 shows when the pre-defined genomic region also comprises an expression cassette for said first DNA Enzyme, located so that upon integration of the donor vector at the pre-defined genomic location it becomes flanked by recognition sites for said second DNA Enzyme and hence can be removed in the presence of said second DNA Enzyme.

FIG. 13 exemplify a variant for targeted integration where a gene editing enzyme is used to catalyze integration of the donor vector into the pre-defined genomic location of the host cell genome.

FIG. 14 shows a variant for targeted integration where recombinase mediated cassette exchange (RMCE) is used to catalyze integration at the pre-defined genomic location of the host cell genome.

FIG. 15 shows a variant for targeted integration where a single recombinase recognition site pair is used to catalyze integration of the donor vector at the pre-defined genomic location of the host cell.

FIG. 16 show the use of a single recombinase recognition site pair to catalyze integration at the pre-defined genomic location and were the promotor P1 present at the pre-defined genomic location is functionally fused to the 5′-part of a split intron.

FIG. 17a-c shows: a) Transient expression of nine different constructs. Plot show % cells in gate for good transient expression of eGFP and mTagBFP2.; b) The mean fluorescence intensity (MFI) for eGFP expression of cultures in log-phase of constructs where B1, B3 and C1 are excluded; and c) Titer measurement of Fc-fusion protein with CEDEX after batch culture of the constructs where 1, B3 and C1 are excluded.

FIG. 18 shows a correlation plot between average single cell fluorescence and bulk titer measurement of Fc-eGFP.

FIG. 19 is a schematic illustration of the Landing Pad Cell line and Donor Vectors used. FC-eGFPx=FC-eGFP gene cassette variant.

FIG. 20 shows Flow cytometry data of cell populations generated using the four different DNA donor vectors. Density of cells in plots having a concentrated main population is visualized by alternating black and white regions (20% of total cells in each region).

FIG. 21 is a schematic illustration of the Landing Pad Cell line and donor vectors used. FC-eGFPx=Signal Peptide carrying FC-eGFP gene cassette variant with different 3′-UTRs. The TagBFP2 cassette carries a signal peptide and is kept constant in all variants.

FIG. 22 is a comparison of Qp calculated from Biacore 8K+ titer measurements and average eGFP signal from flow cytometry data generated using a cell sample from the log phase of each individual batch culture. Panel (a) shows a direct comparison for each vector variants with values normalized to vector variant 524. Panel (b) shows a correlation graph between Qp and eGFP flow cytometry data.

FIG. 23 shows the design of control DNA Donor Vectors (pGE0506-pGE0508) and a small library of DNA Donor Vectors carrying 29 different codons coding for 18 different amino acids and a stop codon at the position of aa 299 of the Glutamine Synthetase gene.
GSx=Glutamine Synthetase gene variant.
PGK=Promotor.

FIG. 24 shows the growth of single copy integration cell pools (generated using transfection of PhiC31 and donor vectors 506-509 followed by two selection steps) in glutamine free media in the absence of MSX (0 MM MSX) or using 10 μM MSX (10 MM MSX).

FIG. 25 shows a graph of out-growth of single cells cloned from the 10 μM MSX pre-selected pools of SDI GS variants (generated using vectors 506, 507 and 509). Individual clones are categorized as growing if the cell confluence reaches 30% or more (of a well in a 96-well culture plate) at day 14 after single-cell cloning.

FIG. 26 shows a Single cell signal generation by fusion of a fluorescent protein to a protein of interest encoded by a single gene. FP1=Fluorescent protein 1, FP2=Fluorescent protein 2.

FIG. 27 shows a Single cell signal generation by fusion of fluorescent protein(s) to a protein of interest encoded by more than one gene. AAV=Adeno-Associated-Virus, VP=Virus Protein.

FIG. 28 shows a Single Cell signal generation by interaction of surface displayed protein of interest and fluorescently labelled target entities. F1=Fluorescent moiety 1, F2=Fluorescent moiety 2.

### DETAILED DESCRIPTION OF THE INVENTION

The present disclosure will now be described more closely in association with the accompanying drawings and some non-limiting examples.

### Definitions

Details of the present disclosure are set forth below. Although any materials and methods similar or equivalent to those described herein can be used in the practice or testing of the present invention, the preferred materials and methods are now described. All words and terms used herein shall be considered to have the same meaning usually given to them by the person skilled in the art, unless another meaning is apparent from the context.

Compositions “comprising” one or more recited elements may also include other elements not specifically recited.

The singular “a” and “an” shall be construed as including also the plural.

“Expression” is used to mean the production of a protein from a gene and refers herein to and comprises the steps of “the central dogma” i.e. the successive action of transcription, translation and protein folding to reach the active state of the protein.

An “expression vector” as defined herein, is a vector comprising nucleic acid sequences to achieve protein expression from the vector when present in a host cell. The expression vector herein is used e.g. to introduce a specific gene of interest into a cell, to thereafter direct the cell machinery for protein synthesis to produce the protein of interest encoded by the gene of interest. An expression vector can contain an “expression cassette”, said expression cassette containing the nucleic acid sequences to facilitate protein expression. In addition, the vector may contain other nucleic acid sequence elements or components.

A “donor vector” as referred to herein, is a vector, preferably a DNA vector, comprising nucleic acid elements or components for facilitating integration of the vector into the pre-defined genomic location of the isolated eukaryotic host cell. The donor vector carries a nucleic acid sequence facilitating a recombination event with a nucleic acid sequence present in the pre-defined genomic location of the host cell, a nucleic acid sequence of interest optionally encoding a protein of interest, a recognition site for the second DNA enzyme and a nucleic acid sequence encoding a first selection marker. Optionally, it may also contain an expression cassette for a second selection marker. A “donor vector” may sometimes herein also simply be referred to as a “vector”. A “donor vector” may sometimes be in the form of an expression vector such as when the donor vector comprises an expression cassette encoding a second selection marker. More specifically, a donor vector described herein contains at least a nucleic acid sequence I2 for recombination with 11 present in the pre-defined genomic location of the eukaryotic cell. In addition, it comprises a nucleic acid sequence of interest, herein also referred to as a gene of interest (“GOI”) if said nucleic acid of interest encodes a protein of interest. It also comprises a nucleic acid sequence E2 comprising a recognition site for the second DNA enzyme which makes it possible to excise parts of the vector backbone once a stable integration of the donor vector has occurred in the pre-defined genomic location of the host cell. It also contains a nucleic acid sequence encoding a first selection marker (SM1), the expression which will only be activated if the donor vector has been integrated into the correct position in the pre-defined genomic location of the host cell. Finally, the donor vector optionally comprises an expression cassette encoding a second selection marker (SM2). Following action of the second DNA enzyme the second selection marker will only be expressed and possible to detect in a cell if a random integration event of the vector has occurred and is used in the second round of selection of the present method. A donor vector is preferably a DNA donor vector but is not limited thereto. A DNA donor vector is sometimes abbreviated “DDV”.

An “expression cassette” is a nucleic acid component forming part of an expression vector which contains all the elements needed for initiation of transcription and translation of the protein of interest. The gene of interest encoding the protein of interest also forms part of the expression cassette. The expression cassette contains e.g. a promoter, essential for the initiation of transcription, and other sequences facilitation transcription, such as enhancer sequences. Sometimes the term “integration cassette” is used herein which corresponds to the nucleic acid sequences from the donor vector that remains at the pre-defined genomic location after the action of the second DNA enzyme. An “integration cassette” may comprise an “expression cassette”.

Herein “a” gene of interest refers to the nucleic acid components needed to produce a protein of interest and as a protein of interest can comprise multiple polypeptide chains can also refer to multiple genes of interest that are present in the same expression cassette. An expression cassette containing multiple genes of interest can either utilize individual promotors to achieve transcription of individual genes or two or more genes can be transcribed as a common mRNA with individual genes separated by i.e. IRES elements. This is in line with that herein, whenever “a” is used, this may also refer to the plural. An example of when an expression cassette comprises more than one gene of interest is when an antibody is to be expressed from the gene of interest, e.g. wherein a light and a heavy chain antibody component are present as separate genes in the expression cassette.

An “intron” is a nucleic acid sequence of a gene that is removed by RNA splicing once transcribed and during production of the final RNA product. Introns are non-coding regions of an RNA transcript, or the DNA encoding it, which are eliminated by splicing before translation.

Herein, a promotor functionally fused to the 5′-part of a split intron means that the transcription of the 5′-part of the split intron is driven by said promotor. Herein, the 5′-part of a split intron is defined as comprising a splice donor site sequence (such as GT). Herein, the 3′-part of a split intron may be defined as comprising (i) a splice branch site sequence, (ii) a Py-rich sequence region and (iii) a splice acceptor site sequence (such as AG).

Transcription comprises the conversion of DNA to RNA by the cell machinery. A “transcription regulatory sequence” is a segment of a nucleic acid sequence which is capable of increasing or decreasing the final expression of specific genes, i.e. by said sequences being capable of regulating the transcription of said gene. Examples of transcription regulatory sequences are promoters, enhancer and the like.

An untranslated region (“UTR”) refers to either of two sections in each end of a coding sequence on a strand of mRNA. The 5 end is named the 5′ UTR and the 3′ side is named the 3′ UTR.

An upstream open reading frame (uORF), as referred to herein, is an open reading frame (ORF) within the 5′ untranslated region (5′UTR) of an mRNA molecule. uORFs are generally involved in the regulation of eukaryotic gene expression. Translation of the uORF typically inhibits downstream expression of the primary ORF (open reading fram), accordingly when present these cause reductions in protein expression. About half of the human genes contain these regions.

An Internal Ribosome Entry Site (“IRES”) is an RNA element that allows for translation initiation in a cap-independent manner. IRES elements are often referred to as distinct regions of RNA molecules that are able to recruit the eukaryotic ribosome to the mRNA. The location for IRES elements is often in the 5′ UTR region but it can also occur elsewhere in the mRNA.

A “plasmid” is a small circular extra-chromosomal DNA molecule that can replicate independently of the cell and are found in bacteria. Plasmids are often used as vectors for molecular cloning i.e. to transfer and introduce selected DNA to a host cell. Plasmids are built-up from specific and necessary elements and may contain genes that can be homo- or heterologous to the bacterial host cell. Plasmids contain e.g. always an bacterial origin of replication and most often a gene for specific antibiotics resistance.

A “nucleic acid sequence of interest” as referred to herein, may be defined as a nucleic acid sequence that one wishes to integrate into a cell to impact the functionality of said cell. It may comprise a gene of interest (“GOI”) that encodes a protein of interest.

By a “recombinant” protein as mentioned herein, is meant a protein manufactured from an expression cassette introduced into a cell by an expression vector. Techniques for producing recombinant proteins are well-known to the person skilled in the art.

A “promoter” is a region of DNA which initiates transcription of a gene upon the binding of RNA polymerase thereto. Promoters are located near the transcription start sites of a gene.

A “host cell” as referred to herein, relates to a eukaryotic cell which is intended to be or has been transformed by a donor vector as disclosed herein.

An “isolated cell”, “isolated host cell” or “isolated eukaryotic host cell” refers to a cell that has been isolated from its natural environment meaning that it is free from any additional components that may occur in nature and that it is not any longer part of its natural environment.

A cell “phenotype” as mentioned herein refers to a cell's observable (physical) characteristics or traits. The term includes the cell morphology, physical form and/or structure. It may also include its developmental processes, its biochemical and/or physiological properties, its behavior, and/or any products of behavior, such as the production of a protein or a measurable amount thereof.

Herein, a “pre-defined genomic location” also sometimes referred to as a “Landing pad” (abbreviated as “LP”), or rather as a pre-defined genomic location comprising a Landing pad sequence, is intended to refer to a location, or a nucleic acid position, characterized by a particular nucleic acid sequence, in a host cell genome. A pre-defined genomic location may also herein be referred to as a “safe harbor site” and/or as a “recombination site”. At the pre-defined genomic location of the host cell, the recombination event between nucleic acid sequence I1 and I2 facilitated by the presence of the first DNA enzyme will occur, initiating expression of the first selection marker and indicating a successful integration event. Basically, the pre-defined genomic location comprises a nucleic acid sequence comprising a recognition site for a first DNA enzyme, a nucleic acid sequence comprising a recognition site for a second DNA enzyme and a promoter nucleic acid sequence.

Herein, when “targeted integration” is referred to, it is intended to mean the integration or the introduction of a nucleic acid sequence element or component into another nucleic acid element or component facilitating a recombination event between such sequences thereby generating a hybrid sequence from the original sequences. Such an integration event is triggered by the presence of an enzyme recognizing nucleic acid sequences in any one or several of the nucleic acid sequence elements or components forming the basis for the recombination.

A “recognition site for an enzyme” refers to a specific combination of nucleotides in a nucleic acid sequence which combination is recognised by a particular enzyme facilitating the binding of the enzyme thereto and wherein the enzyme will thereafter initiate an action at the recognition site, such as a recombination event between two sequences.

The term “DNA enzyme” referred to herein, is defined as an enzyme that acts on DNA, such as cutting pieces of DNA or cutting and integrating DNA into another DNA sequence. The term includes enzymes such as Crisps/Cas9, recombinases, integrases, nucleases etc., but the present disclosure is not limited thereto.

A “first DNA enzyme” referred to herein can be defined functionally as an enzyme that is responsible, in a method disclosed herein, for integration of the donor vector at the pre-defined genomic location of the host cell. The function of the first DNA enzyme is to introduce, not remove, nucleic acid sequences into the pre-defined genomic region. The first DNA enzyme can be one specific enzyme, or it can be different enzymes, when used in a method disclosed herein. This is e.g. if the integration of the donor vector is sequential, and thereby repeated multiple times, introducing multiple copies/variants of the nucleic acid sequence of interest/donor vector into the pre-defined genomic location of the host cell, or if a reversible integration of nucleic acid sequences of interest is performed. Examples of “first DNA enzymes” for use in the context of the present method are given elsewhere herein.

A “second DNA enzyme” referred to herein can be defined functionally as an enzyme that is responsible, in a method disclosed herein, for excision of a nucleic acid sequence region from the pre-defined genomic location having integrated a donor vector, wherein said nucleic acid sequence region is flanked by specific sequences recognized by the second DNA enzyme. When the second DNA enzyme recognises the sequences, it will cut out the nucleic acid sequence component in between these sequences. Examples of “second DNA enzymes” for use in the context of the present method are given elsewhere herein.

“In the presence of a first DNA enzyme” and/or “in the presence of a second DNA enzyme” means that a first and/or a second DNA enzyme is provided in any form as described herein, e.g. as a protein, expressed from a donor vector, a separate expression vector, an expression cassette present in the genome of a cell, a synthetic mRNA etc. “In the presence” is intended to refer to that the function of the first DNA enzyme and/or the second DNA enzyme is provided in any suitable way disclosed herein.

A “selection marker” referred to herein, is a marker that can indicate that a specific event has occurred, such as e.g. in the present context, that an integration of a donor vector has occurred at the pre-defined genomic location of the host cell (first selection marker). The selection marker is often a fluorescent protein that will be expressed by the host cell once the donor vector has been integrated at the correct site of the host cell genome. The expression of the fluorescent protein can e.g. be detected by FACS (Fluorescence-activated cell sorting). Other possible selection markers are mentioned elsewhere herein.

A “first selection marker”, also abbreviated “SM1” herein, can be defined as a silent, non-active or promoter-less selection marker when present in the donor vector. The first selection marker contains a non-coding stretch that is compatible with a promoter that is present in the pre-defined genomic location. Once the donor vector has been integrated into the correct position in the pre-defined genomic position, the first selection marker can be expressed as it now has a promoter to initiate the transcription. Once the selection marker is expressed, the cell population expressing the first selection marker can be selected as positive for stable integration of the donor vector at the pre-defined genomic position. The first selection marker can also be referred to as a “reporter” herein. Examples of suitable first selection markers are provided elsewhere herein.

A “second selection marker”, also abbreviated “SM2” herein, which is an optional feature of a donor vector disclosed herein, can be defined as a non-silent, active and/or functional selection marker when present in the donor vector. The selection marker is encoded as part of an expression cassette, i.e. the selection marker will be expressed transiently upon entry into the cell and later promote stable expression independently of where in the genome it is introduced. The second selection marker is in most aspects of the method presented herein a negative selection marker meaning that cells expressing this marker is preferably not used for recombinant protein production as these cells have (also) integrated a donor vector elsewhere than at the pre-defined genomic position.

### DETAILED DESCRIPTION

There is provided herein a method utilizing a specific Site-Directed Integration (SDI) system for targeted and detectable integration of a donor vector into a pre-defined genomic location of an isolated eukaryotic cell. In addition, the method allows for the identification of random integration events of donor vectors into other parts of the eukaryotic host cell genome of the host cell than into the pre-defined location.

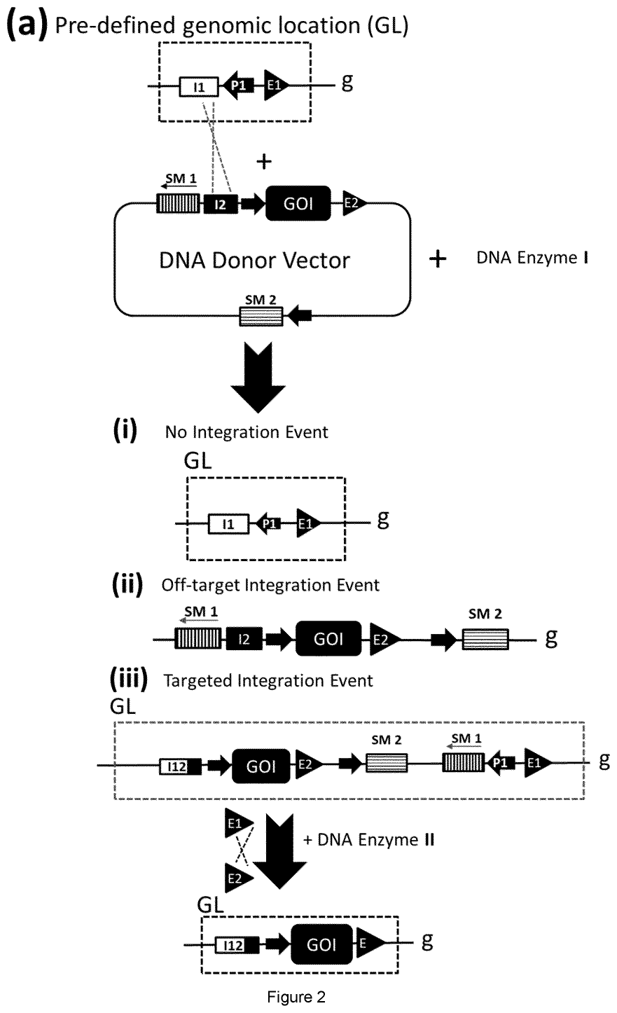

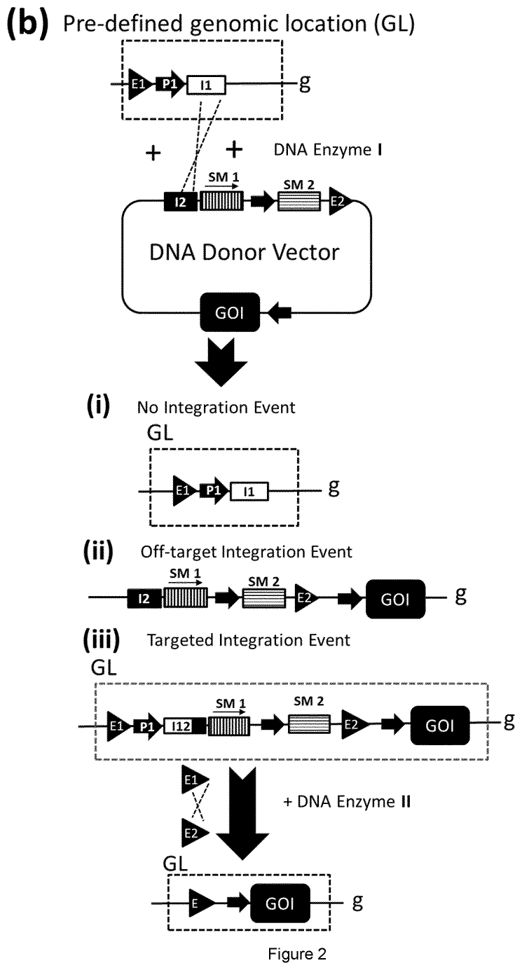

This in combination provides for the optional “double” selection of populations of cells having positively integrated a donor vector at the target site (pre-defined genomic location) of the host cell genome, preferably in the absence of additional random integration of donor vectors at other positions of the host cell genome thereby providing an optimized system for subsequent evaluation of nucleic acid variants impacting recombinant protein expression or recombinant protein function (See FIG. 2). The method in such an aspect uses a combined selection strategy based on a positive (integration at pre-defined genomic location) and a subsequent negative (absence of a random integration event) selection of a population of cells to significantly enrich for cells having integrated a single copy of the insert cassette from the donor vector at the pre-defined genomic location only. This feature is important for applications for which a library of donor vectors comprising nucleic acid variants are integrated into a population of host cells as it ensures a “one to one” correlation between a cellular phenotype and a specific nucleic acid sequence variant and removes biological noise originating from integration at varying genomic locations.

The total solution is based on the integration of so-called “Landing Pad” (LP) sequences at pre-defined genomic locations of the host cell selected for their ability to support high transcription and long-term stability of the same. The Landing pads are designed together with matching donor vectors enabling controlled integration into the pre-defined sites and straight-forward selection of cells in which only the desired integration has occurred. Herein, pre-defined genomic locations and Landing pad/Landing Pad sequences may be used interchangeably.

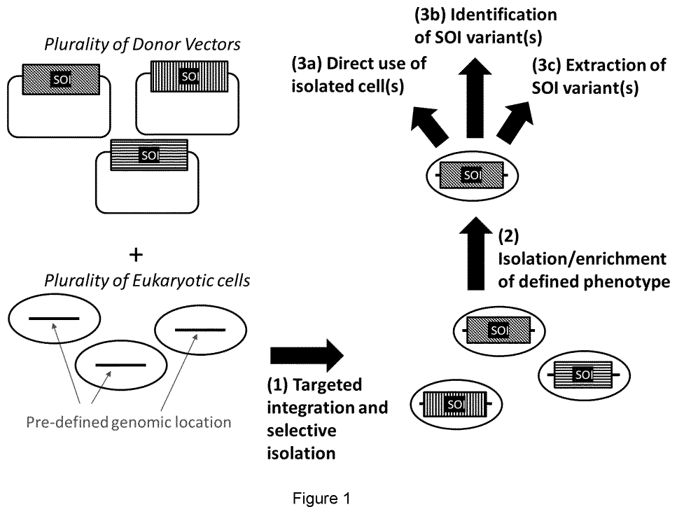

More specifically however, the SDI system is herein used to introduce by targeted integration a library of nucleic acid sequence variants of a nucleic acid sequence into a plurality of host cells from a plurality of donor vectors (See FIG. 1). The system uses a combination of two classes of DNA enzyme recognition sequences together with two different DNA enzymes, for example specific recombinases to enable
(i) integration of a plurality of donor vectors comprising variants of a nucleic acid sequence at a pre-defined genomic location of a plurality of eukaryotic host cells,
(ii) selection of a plurality of cells having integrated a single copy of the donor vector at the pre-defined genomic location using at least one, or possibly two, orthogonal selection steps and
(iii) optionally removal of undesirable sequences from the donor vector at the pre-defined genomic location, and
(iv) a second selection step based on a defined phenotype of a eukaryotic cell having integrated a nucleic acid sequence variant at the pre-defined genomic location.

The SDI method forming part of the full nucleic acid sequence variant optimization method can be performed in different ways all comprising the same general key features.

The general implementation of this SDI method is outlined in FIG. 2. The method is here exemplified based on the use of a single donor vector and a single isolated eukaryotic host cell, however the same principles applies for application to the use of multiple donor vectors (collectively carrying a multitude of nucleic acid sequence variants) for targeted integration into a population of isolated eukaryotic host cells (so that a multitude of nucleic acid sequence variants becomes integrated in different cells).

The pre-defined genomic location of said isolated eukaryotic cell comprises
(i) a nucleic acid sequence I1 comprising a recognition site for a first DNA enzyme,
(ii) a nucleic acid sequence E1 comprising a recognition site for a second DNA enzyme and
(iii) a promotor nucleic acid sequence P1 comprising a start transcription site.

I1, E2 and P1 are configured in either of the two symmetric 5′-3′ sequence orientations O1=[I1, P1 with 3′-5′ directionality, E1] or O2=[E1, P1 with 5′-3′ directionality, I1]. The donor vector comprises:
(i) a nucleic acid sequence I2 promoting recombination with 11 in the presence of said first DNA enzyme,
(ii) a first selection marker gene (SM1) lacking a promotor,
(iii) a recognition site E2 for said second DNA enzyme,
(iv) an Integration Cassette IC and optionally (v) an active expression cassette for a second selection marker gene (SM2).

SM1, SM2 (when present), E2 and IC are configured in either of the two symmetric clockwise orientations O3=[I2, IC, E2, SM2, SM1 with anti-clockwise directionality] or O4=[I2, SM1 with clockwise directionality, SM2, E2, IC]. Nucleic acid sequence elements present in the pre-defined genomic location and the Donor vector are always configured in either of the two matching orientations (a) O1/O3 or (b) O2/O4.

Integration of the full donor vector or parts of the donor vector into the pre-defined genomic location of said isolated eukaryotic cell is achieved by introducing the donor vector into the cell in the presence of a first DNA enzyme, wherein the presence of the first DNA enzyme enables recombination between the nucleic acid sequence I2 of the Donor vector and the nucleic acid sequence I1 present at the pre-defined genomic location of the cell.

Integration at the pre-defined genomic location positions the SM1 gene so that P1 can achieve transcription of the SM1 gene and hence expression of the SM1 gene product. Accordingly, cells having integrated the full donor vector or parts of the donor vector at the pre-defined genomic location can be selected and isolated by using expression of SM1 as a criterion for positive selection.

Optionally, undesirable sequences that can potentially negatively impact the intended functionality of isolated cells can be specifically removed from the pre-defined genomic location in a complementing step leaving only the Integration Cassette (IC) and residual sequences from I1, I2, E1 and E2.

Upon integration of the full donor vector or parts of the donor vector at the pre-defined genomic location, plasmid backbone sequences (i.e. sequences for plasmid propagation in bacteria) as well as expression cassettes for SM1 and SM2 (if present) becomes flanked by the two nucleic acid sequences E1 and E2 (see FIG. 2). In the presence of said second DNA enzyme this sequence region flanked by E1 and E2 is excised from the pre-defined genomic location via the second DNA enzyme acting on E1 and E2. Cells having excised the region flanked by E1 and E2 can be selected and isolated in a negative selection step based on the absence of SM1 expression (if SM2 is not present in the original donor vector) and/or the absence of SM2 expression (if SM2 present in the original donor vector). Besides achieving removal of undesired sequences, this complementing selection step always increases the specificity in isolation of cells having integrated the full donor vector or parts of the donor vector at the pre-defined genomic location as any cell having achieved activation of SM1 (through non-specific mechanisms) after integration outside the pre-defined genomic location will not have SM1 flanked by E1 and E2 and hence will not be selected in a negative selection step based on SM1 expression.

With SM2 present in the donor vector a selection step with improved functionality can be performed following the action of said second DNA enzyme. As SM2 is provided as an active expression cassette, any copy of the donor vector integrated at an undesired genomic location will result in expression of SM2. Importantly however, such integration events will not lead to the SM2 expression cassette being flanked by E1 and E2 as E1 is only present at the pre-defined genomic location. Hence, following the action of said second DNA enzyme leading to excision of sequence regions flanked by E1 and E2, cells having integrated a single copy of the Integration Cassette (IC) at and only at the pre-defined genomic location can be selected and isolated in a negative selection step based on the absence of SM2 expression.

The Integration Cassette (IC) typically comprises an expression cassette for a Gene of Interest (GOI) but applications of the method are not limited thereto.

Specific implementations and further examples of the general SDI method will be exemplified using a single donor vector and a single isolated eukaryotic cell and showing only one of the two possible symmetric orientations of key sequence elements present at the pre-defined genomic location and in the donor vector but is not intended to be limited thereto.

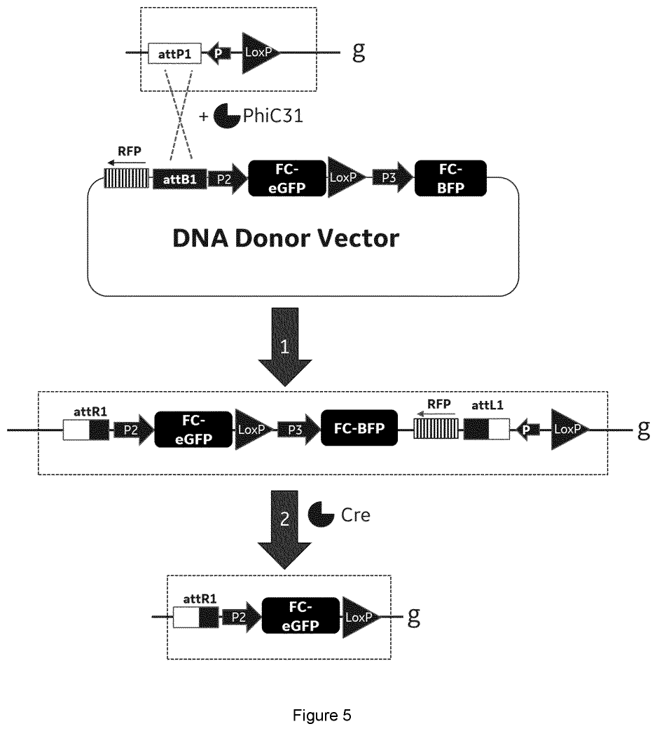

One specific implementation of the design concept is outlined in FIG. 5 featuring an Landing Pad (LP1P1) and a DNA Donor Vector. The results of the experiment performed based on the implementation is also further illustrated and discussed in the experimental section in Example 2. This implementation is merely an example of one way of performing the SDI part of the invention, but it is not intended to be limited thereto. To be clear, for the application to nucleic acid sequence variant optimization a multitude of donor vectors will be specifically integrated into a population of isolated eukaryotic host cells.

Accordingly, in one implementation as illustrated in FIG. 5, the eukaryotic host cell line contains in the pre-defined genomic location a first recombinase recognition sequence (attP1) for the recombinase PhiC31 recombinase, a promotor in 3′ to 5′ orientation and a second recombinase recognition sequence (IoxP) for the recombinase Cre recombinase.

PhiC31 recombinase is a DNA recombinase derived from Streptomyces phage φC31. This enzyme can mediate recombination between two nucleic acid sequences attB and attP. Cre recombinase is also a site-specific recombinase which is used in the present system to subsequently excise the selection system and the plasmid bacterial backbone. Accordingly, the Cre recombinase can be described as “cleaning up” the vector backbone from non-useful sequences once the initial selection has been made. Both PhiC31 recombinase and Cre recombinase are well-known enzymes used in Site Specific Recombination [5, 6].

The matching DNA donor vector includes a first selection marker lacking a promoter (here exemplified by RFP, Red Fluorescent Protein) encoded in anticlockwise orientation, a matching PhiC31 recombinase recognition sequence (attB1), expression cassette(s) comprising a nucleic acid sequence encoding a protein of interest, a complementing recombinase recognition sequence (IoxP) for the Cre recombinase, a fully functional expression cassette for a second selection marker (optional, here exemplified by FC-eGFP) and a plasmid backbone (containing sequences for bacterial propagation etc.).

Co-transfecting the DNA Donor Vector and a vector for expression of PhiC31 into an eukaryotic host cell comprising a pre-defined genomic location of a Landing Pad (LP) sequence, will lead to integration of the donor vector at the LP via PhiC31 mediated recombination of attP1 and attB2 for a fraction of the transfected cells. Upon integration at the pre-defined genomic location, the promoter-less selection marker will be positioned so that it is activated by the promotor in the pre-defined genomic position. Activity of the first selection marker can then be used to select for cells having undergone integration at the LP (using FACS in the case of RFP). Proper selection should generate a pool of cells where most cells have a single copy integrated at the LP. However, a fraction of the cells is expected to have additional copies integrated via off-target integration mechanisms, such as DNA repair mediated random integration and PhiC31 mediated integration at genomic pseudo-attP sequences. To select against such events and at the same time enable removal of selection marker cassettes and plasmid backbone at the pre-defined genomic location (i.e. a “cleaning up”) a second recombinase mediated step has been designed.

Since the pre-defined genomic location contains a IoxP sequence and the DNA Donor Vector also contains a strategically placed IoxP sequence, integration events at the pre-defined genomic location will contain both selection markers (as well as other unwanted sequence elements such as the plasmid backbone), flanked by two IoxP sequences. In contrast, most off-target events should not lead to IoxP flanked selection markers (some random integration events of concatemerized donor vectors could lead to flanked second selection marker genes, but this should be extremely rare). By using a second transfection of a vector encoding the Cre recombinase, the region being flanked by IoxP sequences can be excised from the genome of corresponding cells. Cells having a single copy integrated at the pre-defined genomic location (lacking off-target integration), as well as having unwanted sequence elements removed, can hence be selected for via the absence of selection marker activity (absence of eGFP activity using FACS). This is also called selection by negative selection.

Non-limiting examples of first DNA enzymes for use in a method as defined herein are DNA recombinases, such as a PhiC31 or Bxb1 recombinase, and as described elsewhere herein. A characterizing feature of a recombinase when used as a first DNA enzyme is that it will introduce, not remove, nucleic acid sequence regions into the pre-defined genomic region.

Non-limiting examples of the second DNA enzyme for use in a method as defined herein are DNA recombinases, such as PhiC31 recombinase, Bxb1recombinase, Cre recombinase and Dre recombinase, and as described elsewhere herein. A characterizing feature of a recombinase when used as a second DNA enzyme is that it will remove, not introduce, nucleic acid sequence regions from the pre-defined genomic region.

A nucleic acid sequence I1 comprising a recognition site for a first DNA enzyme may be an attP or an attB site for a PhiC31 or Bxb1 recombinase present in said pre-defined genomic location, or as otherwise exemplified herein depending on which first DNA enzyme is being used in the present context. As an example, it can also be a IoxP site for a Cre recombinase, or as otherwise exemplified herein.

A nucleic acid sequence I2 can be an attB site or an attP site (recognition site) for a PhiC31 or Bxb1 recombinase present in said donor vector, or as otherwise exemplified herein depending on which first DNA enzyme is used in the present context. As an example, it can also be a IoxP site for a Cre recombinase, or as otherwise exemplified herein.

A nucleic acid sequence E1 can be a IoxP site for a Cre recombinase or a roxP site for a Dre recombinase. It can also be an attP or an attB site for a PhiC31 or Bxb1 recombinase, or as otherwise exemplified herein.

A nucleic acid sequence E2 can be a IoxP site for a Cre recombinase or a roxP site for a Dre recombinase. It can also be an attP or an attB site for a PhiC31 or Bxb1 recombinase, or as otherwise exemplified herein.

If the first DNA enzyme is a PhiC31 recombinase, the second DNA enzyme is not a PhiC31 recombinase. The same applies to any other first and second DNA enzymes, i.e. the first and second DNA enzymes are never identical in the same SDI system.

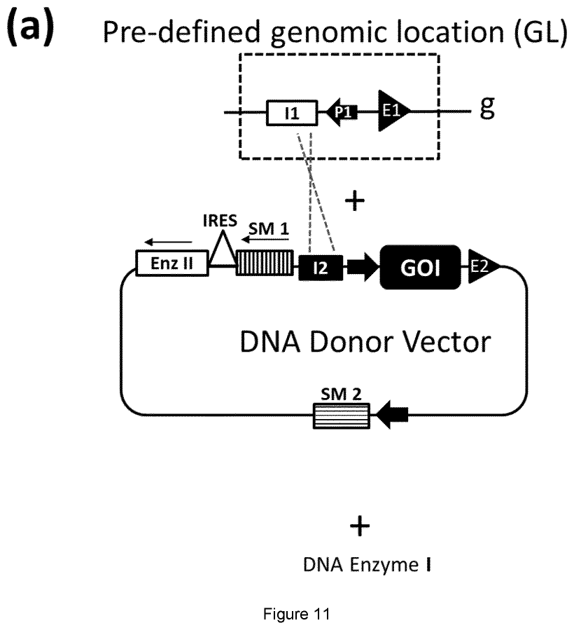

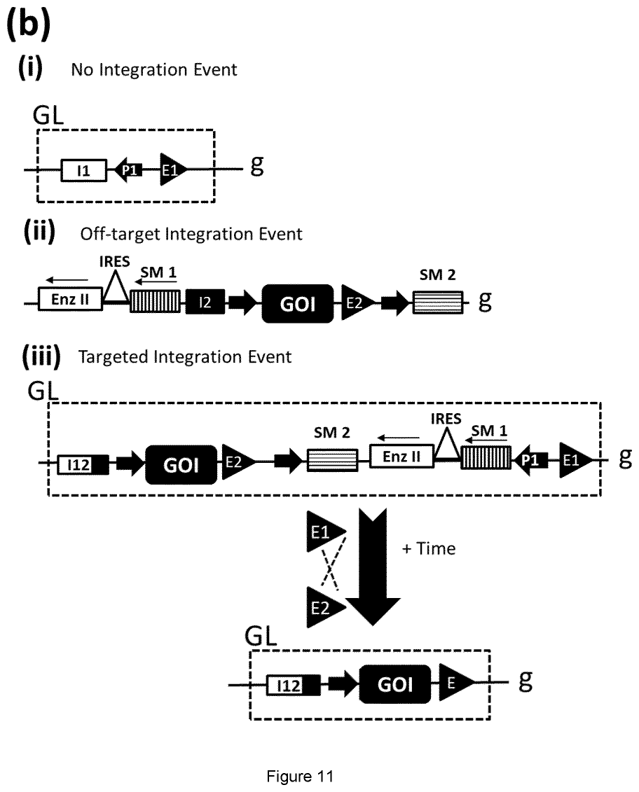

Herein, the first selection marker (SM1) of said donor vector may be linked to a gene coding for said second DNA Enzyme via an IRES element or the amino acid sequences of SM1 and Said second DNA enzyme fused by a self-cleaving peptide such that both the first selection marker and the second DNA Enzyme is activated upon integration at the pre-defined genomic location. This is illustrated in FIG. 11. This ensures presence of the second DNA enzyme once the donor vector has been integrated into the pre-defined genomic location and no further introduction of nucleic acid vectors are needed to proceed with the steps of the method. Expression of SM1 can proceed until the intra-cellular concentration of the second DNA enzyme has reached a high enough value to promote nuclear localization and excision of the sequence region flanked by E1 and E2. By proper timing of the positive selection step, cells having undergone integration at the pre-defined genomic location will contain levels of SM1 allowing positive selection.

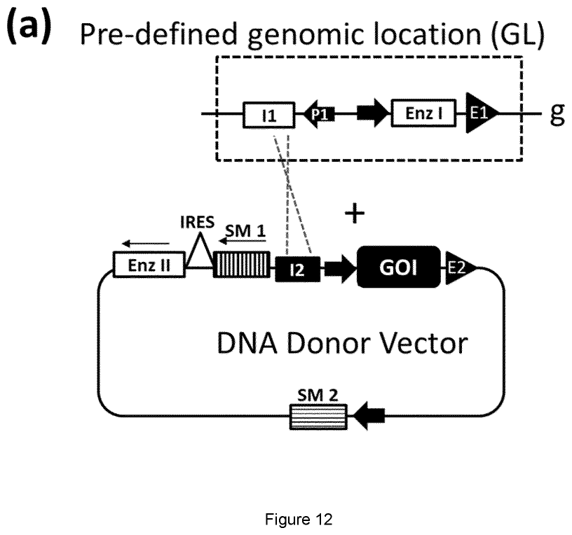

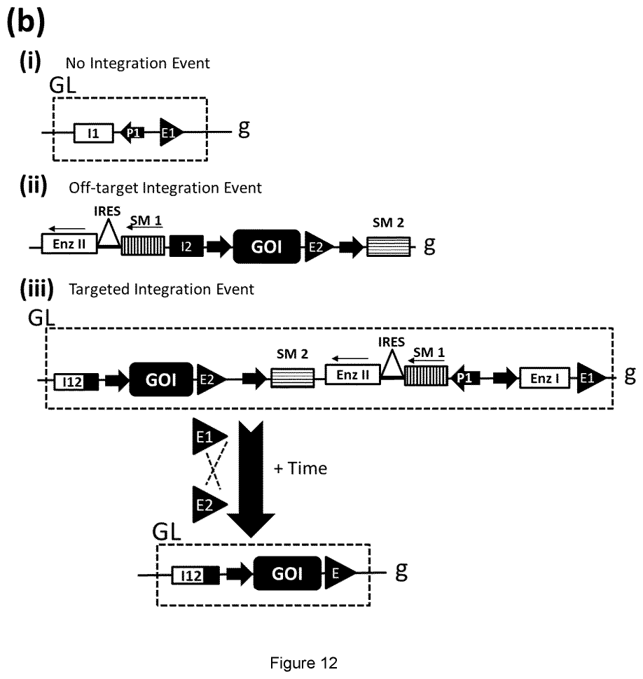

Herein, the pre-defined genomic location may also comprise an expression cassette for said first DNA Enzyme, located so that upon integration of the donor vector at the pre-defined genomic location it becomes flanked by recognition sites for said second DNA Enzyme and excised from the pre-defined genomic region via the action of said second DNA Enzyme. This is illustrated in FIG. 12. This further simplifies the method and should improve the likelihood of high integration efficiencies. Since the expression cassette is removed during later steps of the method, no cellular resources are wasted on expression of the first DNA enzyme in the final isolated cell and any negative consequences of long-term presence of the first DNA enzyme are avoided.

Accordingly, the first DNA enzyme may be provided by expression from the pre-defined genomic location or by introduction into the cell in any form yielding transient presence of said first DNA enzyme in said cell. This includes introduction of an isolated protein perse, the introduction of a separate expression plasmid comprising an expression cassette for said first DNA enzyme, the presence of an active expression cassette for said first DNA enzyme in said donor vector or introduction of a synthetic mRNA encoding said first DNA enzyme.

The second DNA enzyme may be provided as an isolated protein per se, it may be expressed from an expression cassette on a separate expression vector or plasmid or may be expressed from a synthetic mRNA encoding said second DNA enzyme. It may also be expressed from the donor vector once integrated into the pre-defined genomic location as previously described.

As previously mentioned herein, all aspects of the present disclosure allow flexibility in the choice of selection markers without having to make any changes to the pre-defined genomic location. SM1 can be selected from the groups of (i) antibiotic resistance genes, (ii) metabolic enzyme genes such as GS or DHFR, (iii) Fluorescent Protein genes or (iv) Cell surface markers such as CD4 or CD10. SM2 can be selected from the groups of (i) Toxic product generating enzymes such as TK, (ii) Fluorescent Protein genes or (iii) Cell surface markers such as CD4 or CD10.

Preferably both selection markers are selected from the groups of (i) Fluorescent protein genes or (ii) Cell surface markers allowing fast selection steps via methods such as FACS or MACS.

The expression of the first or second selection marker can be detected e.g. by using FACS, if the selection marker is a Fluorescent protein. If the selection marker is an antibiotic resistance gene, the integration can be detected by culturing cells in the presence of the corresponding antibiotic. If the cells survive in a media to which an antibiotic has been added, the donor vector has been successfully integrated.

As mentioned herein, the recombinases are useful for excising nucleic acid sequences flanked by the appropriate nucleic acid regions (E1 and E2) in the host cell genome. This is a step mainly to “tidy up” in the host cell genome as some parts of the nucleic acid sequence introduced into the pre-defined genomic location will be superfluous once an integration and selection has been made. Their presence may also consume cell energy. Excising of a nucleic acid sequence means that the second DNA enzyme by binding to specific combinations of nucleotides, i.e. E1 and E2, is capable of cutting and removing nucleic acid sequence parts from the host cell genome. The presence of the nucleic acid sequences E1 and E2 at the pre-defined genomic location is in principle proof of that a stable integration of the donor vector has occurred there.

The expression cassette encoding the second selection marker (if present) is positioned in said donor vector, so that upon integration at the pre-defined genomic location it becomes flanked by E1 and E2. However, if the donor vector is integrated outside the pre-defined genomic location, said expression cassette encoding a second selection marker will not be flanked by E1 and E2.

Accordingly, if it is possible to detect, e.g. by FACS, expression of a second selection marker (SM2) among cells in the cell population following action of the second DNA enzyme, this means that an undesired integration event of a donor vector has occurred at another position in the cell. Such cells could be removed to select for (by negative selection) the cells where integration of the donor vector has occurred only at the pre-defined genomic location.

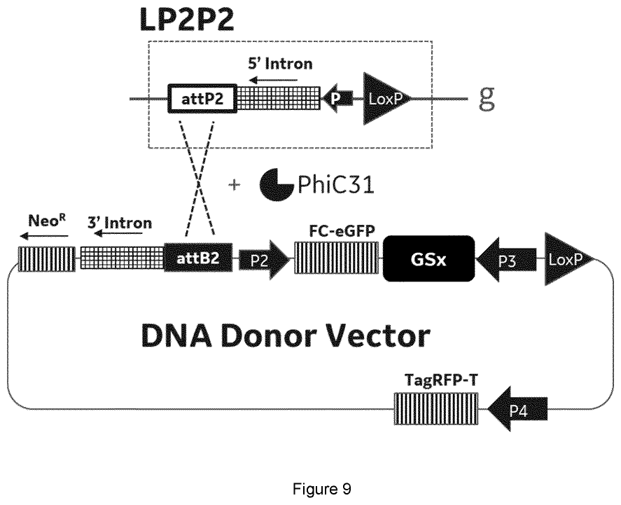

In FIG. 9 and in Example 4 it is illustrated the generation of an SDI cell pool using two consecutive selection steps, i.e. where a second DNA enzyme is added after the integration to remove nucleic acid sequences that do not fulfil a purpose in the cell any longer. In this example, an antibiotic resistance gene was used as a first selection marker (SM1). A second round of selection was performed using Cre recombinase to excise nucleic acid sequences flanked by the IoxP nucleic acid regions in each end. The presence of a random integration event was detected by a double positive signal of GFP/RFP (Green/Red Fluorescent Protein) using FACS. The cells were sorted based on the positive/negative GFP/RFP signal. This additional step provides for the removal of cells that may have integrated one donor vector at the pre-determined genomic location but that may also have randomly integrated a second or further donor vector(s) at a random non-target position(s) in the host cell genome.

As previously mentioned herein, said first DNA enzyme may be a recombinase. The first DNA enzyme may be a mix of different DNA enzymes, such as recombinases, as long as any of the DNA enzymes of the first DNA enzyme is not the same as the second DNA enzyme.

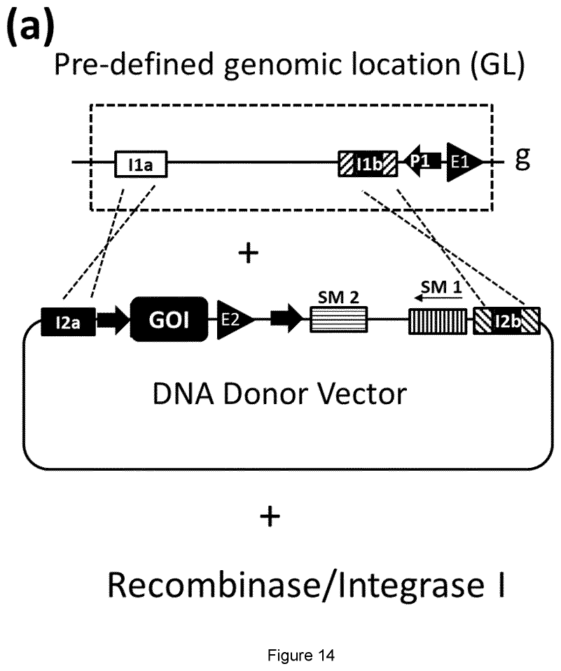

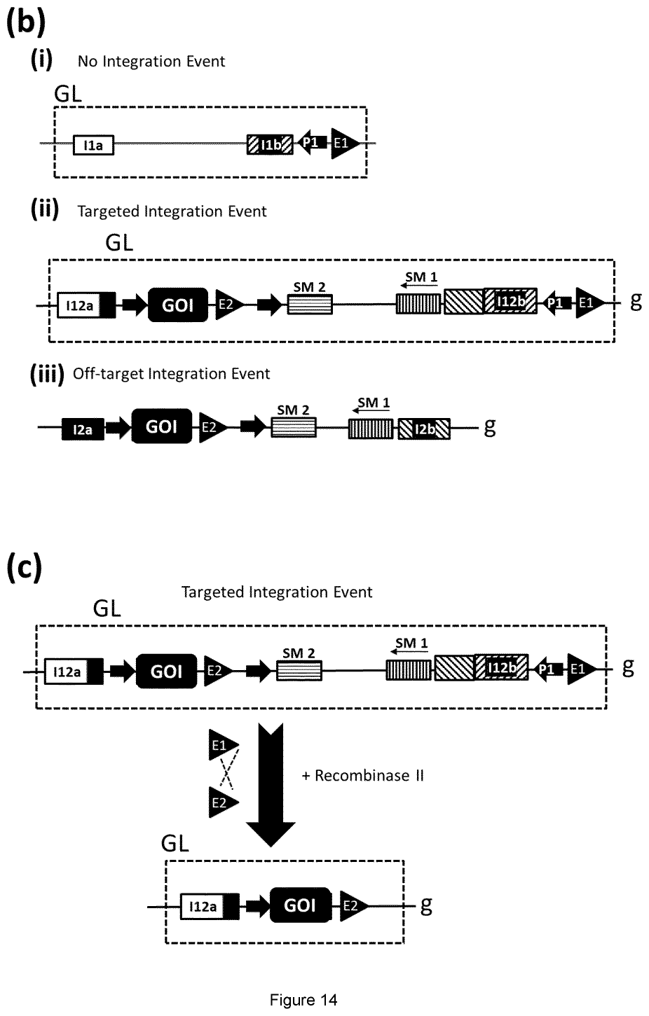

Herein, there may be more than one recognition site for said first DNA enzyme present in the donor vector, such as two or more recognition sites. This means that there will also be more than one recognition site for said first DNA enzyme present in the pre-defined genomic location, such two or more recognition sites. An example of such a system utilizing recombinases is shown in FIG. 14. FIG. 14 show a recombinase mediated cassette exchange (RMCE) to catalyze integration at the pre-defined genomic location. Variants thereof modified according to FIG. 2, FIGS. 11-12 and FIG. 16 are also encompassed by the present disclosure.

Hence, in the example in FIG. 14, the pre-defined genomic location comprise in 5′-3′ sequence order; (i) a first recognition site for a first recombinase enzyme (I1A); (ii) a second recognition site for said first recombinase enzyme (I1B), (iii) a Promotor P1 with 3′-5′ directionality and (v) a recognition site E1 for a second recombinase enzyme.

In this example, the donor vector comprise in 5′-3′ sequence order; (i) a third recognition site for said first recombinase enzyme (I2A), (ii) an Integration Cassette (IC), here exemplified by an expression cassette for a Gene of Interest (GOI), (iii) a recognition site E2 for said second recombinase enzyme, (iv) an expression cassette for a second Selection Marker (SM2), (v) a gene for a first Selection Marker (SM1) encoded in 3′-5′ directionality and (vi) a fourth recognition site for said first recombinase enzyme (I2B).

Introduction of the donor vector and a first recombinase into a population of cells results in; (a) integration of the Integration cassette in the donor vector, i.e. the sequence region flanked by said third and fourth recombinase recognition sites at the pre-defined genomic location for a fraction of the cells (See FIG. 14b, panel (ii)) and (b) off-target genomic integration (outside the pre-defined genomic location) of the donor vector for a fraction of cells (See FIG. 14b, panel (iii)).

Integration at the pre-defined genomic location results in the formation of an active expression cassette for SM1 (See FIG. 14b, panel (ii)). Further, following integration at the pre-defined genomic location both SM1 and SM2 are flanked by two recognition sites for said second recombinase enzyme.

Integration by an off-target event (See FIG. 14b, panel (iii)) does not typically lead to the activation of SM1 but does integrate an active SM2 that is not flanked by two recognition sites for said second recombinase enzyme.

Cells having undergone integration at the pre-defined genomic location differs from cells with no integration event (See FIG. 14b, panel (i)) and cells having undergone only an off-target integration event through the activity of SM1. Hence, activity of SM1 can be used to select for cells having undergone integration at the LP.

To remove cells having undergone an off-target integration event in addition to integration at the pre-defined genomic location, recombinase activity of said second recombinase is introduced within cells selected for SM1 activity. For integration at the LP, this results in the excision of both SM1 and SM2 and hence their corresponding activity. For off-target integration events this reaction cannot occur and SM2 activity remains. As a result, cells having undergone only the desired targeted integration event at the pre-defined genomic location can be selected from cells having undergone a multiple integration event through absence of SM2 activity.

The pre-defined genomic location for the finally selected Cells (See FIG. 14c) does not contain the expression cassette for SM2 nor the activated expression cassette for SM1 or any residual sequence from the donor vector except a sequence created through the recombination of E1 and E2 (E).

Said first recombinase enzyme can be selected from the groups of (i) Serine recombinases or (ii) Tyrosine recombinases.

Said first to fourth recombinase recognition sites can be selected according to;
(a) I1a=I1b and I2a=I2b using one matching recognition site pair for serine recombinases such as PhiC31 [I1a=I1b=attP or attB and I2a=I2b=attB or attP] or Bxb1 [I1a=I1b=Bxb1 attP or Bxb1 attB and I2a=I2b=Bxb1 attB or Bxb1 attP],
(b) Selecting two different matching recognition site pairs using mutated recognition pairs for serine recombinases such as PhiC31 (I1a and I1b=different attP variants or attB variants; I2a and I2b=different attB variants or attP variants) and
(c) I1a=I2a and I1b=I2b for tyrosine recombinases, were mutated recognition site variant pairs exist, such as Cre (LoxP1 and LoxP2 selected from available mutated IoxP pairs), Dre (rox1 and rox2 selected from available mutated rox pairs) or FLP (FRT1 and FRT2 selected from available mutated FRT pairs).

Said second recombinase enzyme is different from said first recombinase enzyme and can be selected from the groups of (i) Serine recombinases or (ii) Tyrosine recombinases.

Recognition sites E1 and E2 for said second recombinase enzyme can be identical in sequence as exemplified by; (i) E1=E2=IoxP or its mutated variants for use with the Cre recombinase, (ii) E1=E2=rox or its mutated variants for use with the Dre recombinase or (iii) E1=E2=FRT or its mutated variants for use with the FLP (Flippase) recombinase.

Recognition sites E1 and E2 for said second recombinase enzyme can have different sequences as exemplified by; (i) E1=attP and E2=attB or its mutated variants for use with the PhiC31 recombinase or (ii) E1=Bxb1 attP and E2=Bxb1 attB or its mutated variants for use with the Bxb1 recombinase.

The further recognition sites for the first DNA enzyme can be referred to herein as variants of I1, i.e. I1a and I1b, and variants of I2, i.e. I2a and I2b, and so on.

Accordingly, there is also provided herein a method herein, wherein
(a) I1 comprises two recombinase recognition site variants I1a and I1b; and
(b) I2 comprises two recombinase recognition site variants I2a and I2b; and
(c) I1a is capable of recombination with I2a and I1b is capable of recombination with I2b in the presence of said first DNA enzyme.

Sometimes, I1a is identical to I2a and I1b is identical to I2b. I1a, I1b, I2a and I2b may be selected from IoxP, rox or FRT or variants thereof, respectively, and the first DNA enzyme may be selected from the group consisting of a Cre recombinase, a Dre recombinase and a FLP recombinase [5], respectively.

Herein, there is also provided a method, wherein:
(a) I1 comprises a single recombinase recognition site; and
(b) I2 comprises a single recombinase recognition site; and
(c) I1 and I2 are capable of recombination in the presence of said first DNA enzyme.

The recombinase recognition site comprised by I1 may also differ in sequence from the recombinase recognition site comprised by I2. The recombinase recognition sites provided herein may be selected from attB, attP, Bxb1 attP, Bxb1 attB or a variant thereof. Said recombinase may be a PhiC31 or Bxb1 recombinase or a mutant thereof.

Any variant or mutant of a recognition site/DNA enzyme will be a functionally equivalent variant or mutant thereof. The skilled person will construct and produce such a functionally equivalent variant or mutant.

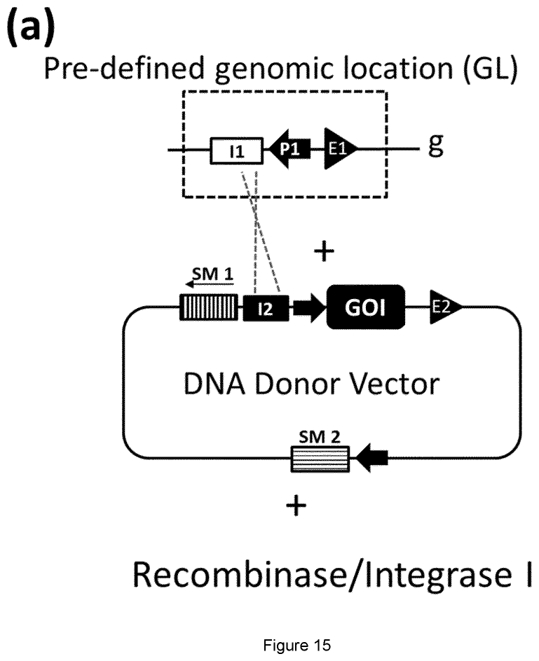

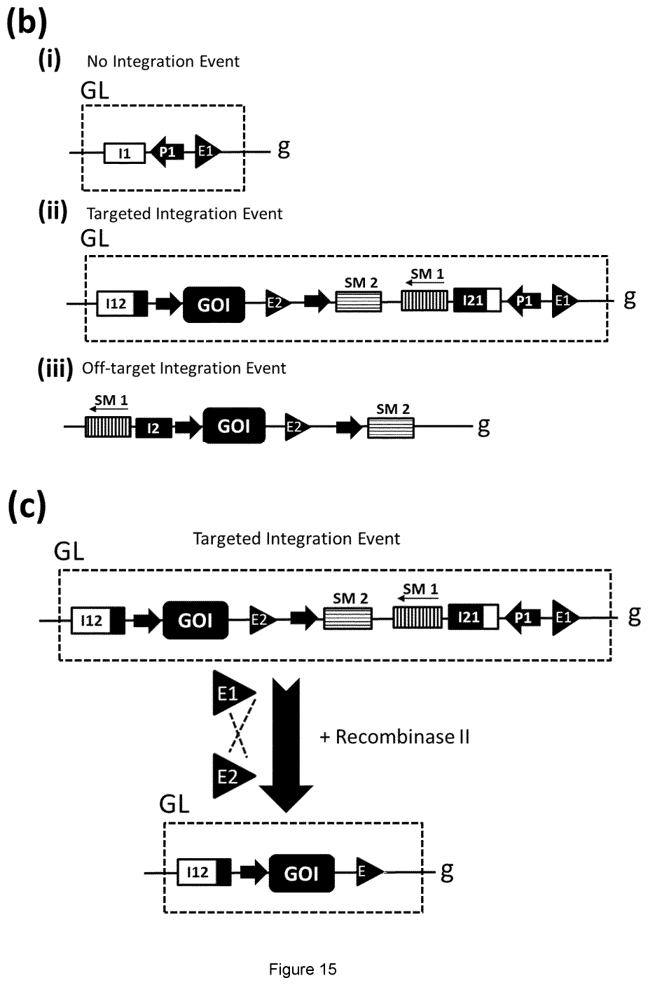

An example of using a single recombinase recognition site pair to catalyze integration at the pre-defined genomic location is shown in FIG. 15. Variants modified according to FIG. 2, FIG. 11-12 and FIG. 16 are also encompassed by this disclosure.

In this example, the pre-defined genomic location comprise in 5′-3′ sequence order; (i) a first recognition site for a first recombinase enzyme (I1); (ii) a Promotor P1 with 3′-5′ directionality and (iii) a first recognition site E1 for a second recombinase enzyme.

In this example, the donor vector comprise in 5′-3′ sequence order; (i) a second recognition site for said first recombinase enzyme (I2), (ii) an Integration Cassette (IC), here exemplified by an expression cassette for a Gene of Interest (GOI), (iii) a second recognition site E2 for said second recombinase enzyme, (iv) an expression cassette for a second Selection Marker (SM2) and (v) a gene for a first Selection Marker (SM1) encoded in 3′-5′ directionality.

Introduction of the donor vector and a first recombinase into a population of LP Cells results in; (a) integration of the donor vector at the LP for a fraction of the LP cells (See FIG. 15b, panel (ii)) and (b) off-target genomic integration (outside pre-defined genomic location) of the donor vector for a fraction of the cells (See FIG. 15b, panel (iii)).

Integration at the pre-defined genomic location results in the formation of an active expression cassette for SM1 (See FIG. 15b, panel (ii)). Further, following integration at the pre-defined genomic location both SM1 and SM2 are flanked by the two recognition sites E1 and E2 for said second recombinase enzyme.

Integration by an off-target event (See FIG. 15b, panel (iii)) does not typically lead to the activation of SM1 but does integrate an active SM2 that is not flanked by recognition sites for said second recombinase enzyme.

Cells having undergone integration at the pre-defined genomic location differs from cells with no integration event (See FIG. 15b, panel (i)) and cells having undergone only an off-target integration event through the activity of SM1. Hence, activity of SM1 can be used to select for cells having undergone integration at the pre-defined genomic location.

To remove cells having undergone an off-target integration event in addition to integration at the pre-defined genomic location, recombinase activity of said second recombinase is introduced within cells selected for SM1 activity. For integration at the pre-defined genomic location, this results in the excision of both SM1 and SM2 and hence their corresponding activity. For off-target integration events this reaction cannot occur and SM2 activity remains. As a result, cells having undergone only the desired targeted integration event at the LP can be selected from LP Cells having undergone a multiple integration event through absence of SM2 activity.

The pre-defined genomic location for the finally selected cells (See FIG. 15c) does not contain the expression cassette for SM2 nor the activated expression cassette for SM1 or any residual sequence from the donor vector except sequences created through the recombination of I1 and I2 (I12) and E1 and E2 (E).

Said first recombinase enzyme can be selected from the group of Serine recombinases such as PhiC31 and Bxb1. Said first and second recombinase recognition sites (I1 and I2) can be selected with recognition sites matching chosen recombinase according to; (a) I1=attP variant and I2=attB variant or (b) I1=attB variant and I2=attP variant.

Said second recombinase enzyme is different from said first recombinase enzyme and can be selected from the groups of (i) Serine recombinases or (ii) Tyrosine recombinases.

Recognition sites E1 and E2 for said second recombinase enzyme can be identical in sequence as exemplified by; (i) E1=E2=IoxP or its mutated variants for use with the Cre recombinase, (ii) E1=E2=rox or its mutated variants for use with the Dre recombinase or (iii) E1=E2=FRT or its mutated variants for use with the FLP recombinase.

Recognition sites E1 and E2 for said second recombinase enzyme can have different sequences as exemplified by; (i) E1=attP and E2=attB or its mutated variants for use with the PhiC31 recombinase or (ii) E1=Bxb1 attP and E2=Bxb1 attB.

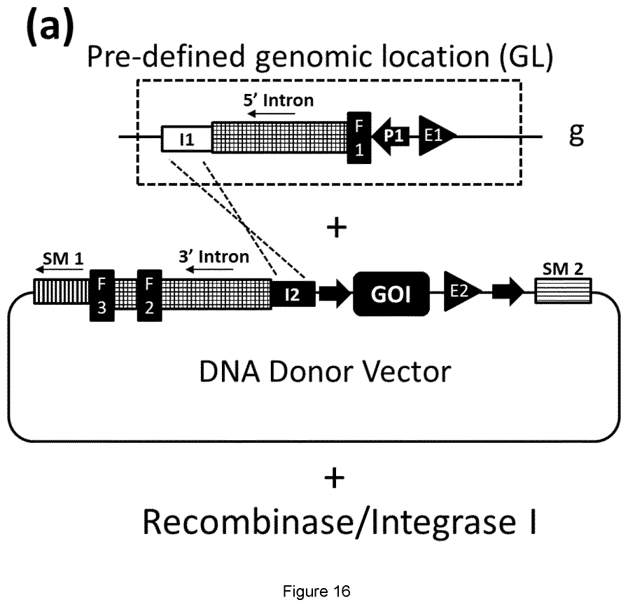

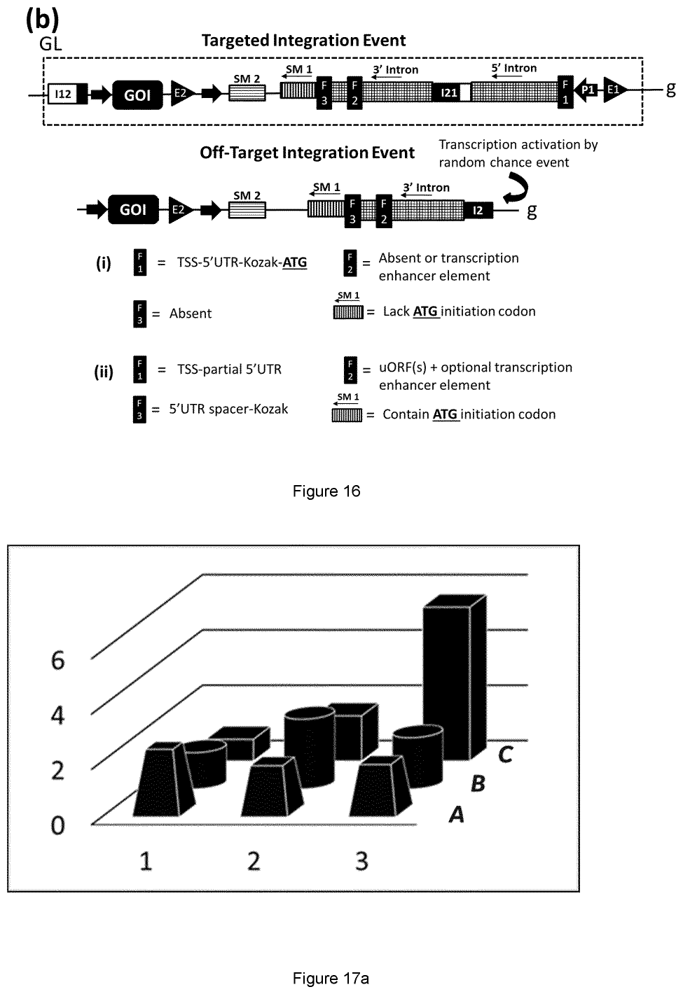

There is also provided a method, as illustrated in FIG. 16, which exemplify using a single recombinase recognition site pair to catalyze integration at the pre-defined genomic location and wherein the promotor P1 present at the pre-defined genomic location is functionally fused to the 5′-part of a split intron. Methods described earlier but modified according to FIG. 16 (i.e. using a split intron design) are also encompassed by the present disclosure.

In this example the pre-defined genomic location further comprises the 5′-part of an Intron with 3′-5′ directionality and a functional sequence region F1 with 3′-5′ directionality between said first recognition site for said first recombinase enzyme and said promotor P1 with 3′-5′ directionality.

In this example the donor vector further comprise a sequence region located between said first selection marker SM1 with 3′-5′ directionality and said second recognition site for said first recombinase enzyme. Said sequence region comprise in 5′-3′ sequence order; (a) a functional sequence region F3 with 3′-5′ directionality and (b) the 3′-part of an Intron with 3′-5′ directionality further comprising a functional sequence region F2 downstream of the splice acceptor site sequence.

Upon integration at the pre-defined genomic location (See FIG. 16b, upper panel) a complete expression cassette, including a functional Intron, for said first selection marker SM1 is formed. Hence expression of SM1 is activated.

Following an off-target integration event (See FIG. 16b, lower panel) a truncated version of the SM1 expression cassette is integrated.

By chance events transcription of the truncated SM1 cassette can occur. This can be due to
(a) promotor rescue in which the donor vector is integrated in such a way that the truncated SM1 cassette becomes located in frame with a native promotor present in the cell genome or
(b) cleavage and concatamerization of the donor vector such that a promotor present in the donor vector becomes re-oriented in frame with the truncated SM1 cassette followed by integration of the resulting concatamer.

Such chance events can reduce specificity in SM1 based selection of cells having integrated the donor vector at the pre-defined genomic location. By use of specific combinations of functional sequence regions F1-F3, improved specificity can be achieved (See FIG. 16b).

In a first design of F1-F3; (a) SM1 (when present in the donor vector) lacks the ATG start codon and is directly fused to the 3′-Intron, (b) F1 is made up of (from 3′-5′) a start transcription site (TSS), a first 5′-UTR region, a Kozak/translation initiation site and an ATG start codon all with 3′-5′ directionality. Following an off-target integration event this means that any SM1 gene integrated will lack a start codon and hence will not generate expression of a functional SM1 protein. However, upon integration at the pre-defined genomic location a functional expression cassette will be formed. Upon splicing of the Intron, the ATG start codon will be directly fused to SM1, leading to proper expression of SM1 protein.

In a second design of F1-F3; (a) SM1 contains an ATG start codon, (b) F3 is made up of (from 3′-5′) a second 5′-UTR region and a Kozak/translation initiation site all with 3′-5′ directionality, (c) F2 comprise at least one short upstream open reading frame (uORF) with 3′-5 directionality and (d) F1 is made up of a start transcription site (TSS) and a first 5′-UTR region. Following an off-target integration event the truncated SM1 cassette will typically retain the one or more uORFs. These uORFs will reduce initiation at the intended SM1 start codon thereby improving discrimination between off-target integration based SM1 activation and SM1 activation based on integration at the pre-defined genomic location. Preferably, multiple uORFs in series are used and placed with minimal distance to the SM1 start codon (directly downstream of the Intron Splice branch site).

The use of a split intron design also improves the expression of activated SM1 as optimal 5′-UTR sequences can be used for SM1. In designs lacking a split intron, a sequence generated through recombination of I1 and I2 (see FIG. 15) will be comprised by the SM1 5′-UTR. This leads to an extended 5′-UTR with potentially non-optimal sequence composition that can reduce the obtainable expression level of SM1 (impacting specificity in positive selection steps based on SM1). Via the use of a split intron as described herein, the I1/I2 recombination product becomes incorporated in the fully formed intron upon integration at the pre-defined genomic location (See FIG. 16). Upon generation of mature SM1 mRNA by the cell, the intron is spliced out and the corresponding SM1 5′-UTR fully defined by F1 and F2. Accordingly, the SM1 5′-UTR can be design with full control to optimize SM1 expression for an intended purpose. Variations in the design of F2-F3 further offers flexibility in the expression level of SM1 upon integration at the LP. Increasing the length of the 5′-UTR region of F3 reduces expression of SM1 and addition of a transcription enhancer element in F2 can increase expression of SM1 above what can be achieved with an optimal 5′-UTR only.

Finally, the use of a split intron design can improve the efficiency of recombination between I1 and I2 at the pre-defined genomic location as shown in the experimental section, Example 1. One potential explanation to the improved integration efficiency observed is that the 5′-part of the split intron at the pre-defined genomic location function as a critical spacer that can avoid/reduce steric interference between RNA polymerase initiation complex binding around the start transcription site and copies of the first DNA enzyme (for example PhiC31) performing its function by binding and manipulation of I1. To increase integration efficiency, said 5′-part of a split intron can be designed to have a length of at least 50 bp, at least 100 bp or at least 300 bp.

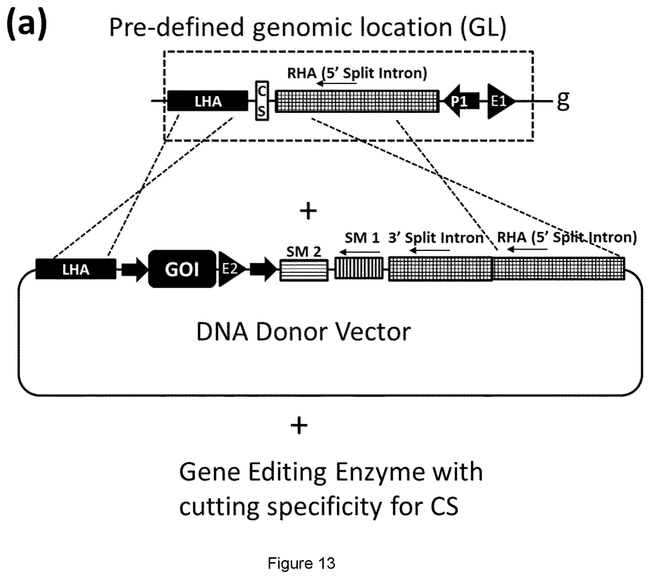

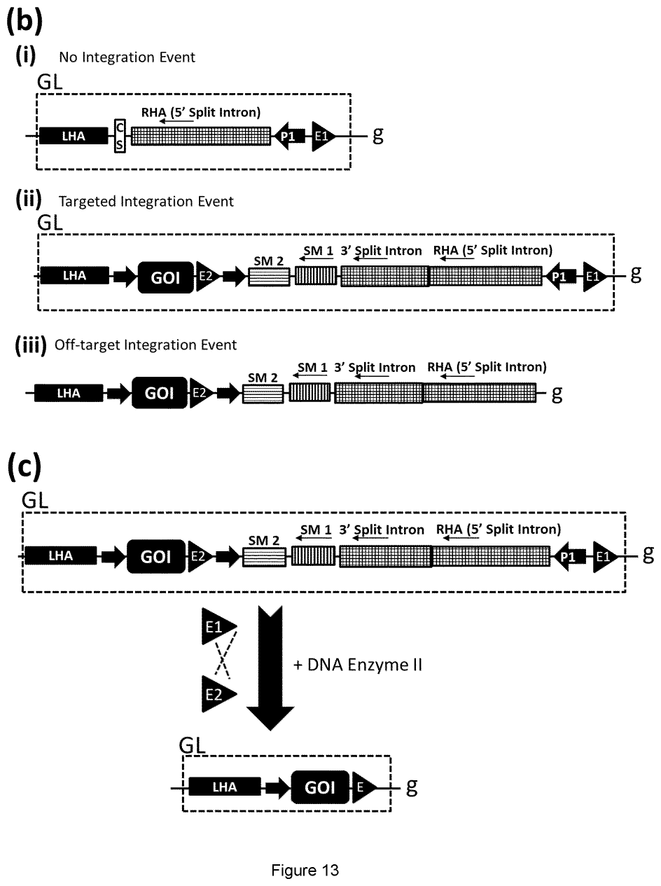

FIG. 13 illustrate a method wherein a gene editing enzyme is used to catalyze integration of the donor vector at the pre-defined genomic location (the first DNA enzyme is a gene editing enzyme). Modifications thereof according to FIG. 2 and FIG. 11-12 are also encompassed by the present disclosure.

In this example the pre-defined genomic location comprise in 5′-3′ sequence order; (i) a Left Homology arm (LHA), (ii) a recognition site/Cut Site (CS) for the gene editing enzyme, (iii) a Right Homology Arm (RHA) which also function as the 5′-part of an Intron with 3′-5′ directionality (i.e. having a splice donor Site at the end closest to the promotor), (iv) a Promotor P1 with 3′-5′ directionality and (v) a recognition site E1 for a second DNA enzyme.

In this example, the donor vector comprise in 5′-3′ sequence order; (i) said Left Homology Arm (LHA), (ii) an Integration Cassette (IC), here exemplified by an expression cassette for a Gene Of Interest (GOI), (iii) a recognition site E2 for said second DNA enzyme, (iv) an expression cassette for a second Selection Marker (SM2), (v) a gene for a first Selection Marker (SM1) encoded in 3′-5′ directionality, (vi) the 3′-part of an Intron with 3′-5′ directionality (i.e. having a splice branch site and a splice acceptor site at the end closest to SM1) and (vii) said Right Homology Arm (RHA) which also function as the 5′-part of an Intron with 3′-5′ directionality.

Introduction of the donor vector and a gene editing enzyme with cut specificity for CS into a population of eukaryotic cells results in; (a) a double strand break at CS in the pre-defined genomic location for a fraction of the eukaryotic cells, (b) Integration of the donor vector region flanked by the LHA and RHA by Homology Directed DNA Repair for a fraction of eukaryotic cells having a double strand break at CS, (c) off-target genomic integration (outside the pre-defined genomic region) of the donor vector for a fraction of LP cells.

Integration at the pre-defined genomic location results in the formation of an active expression cassette for SM1 (See FIG. 13b, panel (ii)). As the integration event further creates a fully functional Intron between the Promotor P1 and SM1, mature mRNA for SM1 does not comprise the RHA. Further, following integration at the LP both SM1 and SM2 are flanked by two recognition sites for said second DNA Enzyme.

Integration by an off-target event (See FIG. 13b, panel (iii)) does not typically lead to the activation of SM1 but does integrate an active SM2 that is not flanked by two recognition sites for said second DNA Enzyme.

Eukaryotic cells having undergone integration at the pre-defined genomic location differs from cells with no integration event (See FIG. 13b, panel (i)) and cells having undergone only an off-target integration event through the activity of SM1. Hence, activity of SM1 can be used to select for cells having undergone integration at the pre-defined genomic location.

To remove cells having undergone an off-target integration event in addition to integration at the pre-defined genomic location, recombinase activity (second DNA Enzyme) capable of recombining E1 and E2 is introduced within the cells selected for SM1 activity. For integration at the LP, this results in the excision of both SM1 and SM2 and hence their corresponding activity. For off-target integration events this reaction cannot occur and SM2 activity remains. As a result, the cells having undergone only the desired targeted integration event at the pre-defined genomic location can be selected from cells having undergone a multiple integration event through absence of SM2 activity.

The pre-defined genomic location for the finally selected cells (See FIG. 13c) does not contain the expression cassette for SM2 nor the activated expression cassette for SM1 or any residual sequence from the donor vector except a sequence created through the recombination of E1 and E2 (E).

The gene editing enzyme may be selected from the groups of (i) Zinc Finger Nucleases (ZFNs); Homing Endo Nucleases such as Meganucleases; (iii) TALENs or (iv) DNA or RNA guided nucleases, such as CRISPR/Cas9, but it is not limited thereto.

Said second DNA Enzyme has recombinase activity and may be selected from the groups of (i) Serine recombinases or (ii) Tyrosine recombinases.

E1 and E2 may be identical in sequence as exemplified by; (i) E1=E2=IoxP or its mutated variants for use with the Cre recombinase, (ii) E1=E2=rox or its mutated variants for use with the Dre recombinase or (iii) E1=E2=FRT or its mutated variants for use with the FLP recombinase.

E1 and E2 can have different sequences as exemplified by; (i) E1=attP and E2=attB for use with the PhiC31 recombinase or (ii) E1=Bxb1 attP and E2=Bxb1 attB.

Accordingly, there is provided herein a method, wherein said first DNA enzyme is a gene editing enzyme, such as a gene editing nuclease. Thereby there is provided a method, wherein: (a) I1 comprises a cut site for said gene editing nuclease and two sequence regions LHA1 and RHA1; and (b) I2 comprises two sequence regions LHA2 and RHA2 homologous to LHA1 and LHA2; and (c) I1 and I2 are capable of recombination in the presence of said first DNA enzyme.

As previously mentioned, there is provided a method wherein said gene editing enzyme is selected from the group consisting of (i) zink finger nucleases (ZFNs); (ii) homing endo nucleases, such as meganucleases; (iii) TALENS and (iv) DNA or RNA guided nucleases, such as CRISPR/Cas 9, but the present disclosure is not limited thereto.

The nucleic acid sequences E1 and E2 may be identical recombinase recognition sites, such as IoxP, rox or FRT or variants thereof, respectively, provided that E1 and E2 are different from I1 and I2.

Said second DNA enzyme may be selected from the group consisting of a Cre recombinase, a Dre recombinase and a FLP recombinase, provided that the first DNA enzyme is not a Cre recombinase, a Dre recombinase or a FLP recombinase.

In a method provided herein, the promotor nucleic acid sequence P1 when integrated at said pre-defined genomic location may functionally be fused to the 5′-part of a split intron. This is illustrated in FIG. 16 previously discussed herein. The introduction of a split intron between the promoter P1 and I1 (or a variant thereof) in the pre-defined genomic location provides a “spacer” minimizing steric hindrance which may occur due to blockage from the polymerase to the promoter. The presence of this spacer provides for an improved expression of the first selection marker (SM1) as shown in the experimental section, Example 1.

Accordingly, there is provided herein a method wherein the pre-defined genomic location further comprises the 5′-part of an Intron with 3′-5′ directionality and a functional sequence region F1 with 3′-5′ directionality between said first recognition site for said first recombinase enzyme and said promotor P1 with 3′-5′ directionality and wherein the donor vector further comprise a sequence region located between said first selection marker SM1 with 3′-5′ directionality and said second recognition site for said first recombinase enzyme. Said sequence region comprise in 5′-3′ sequence order; (a) a functional sequence region F3 with 3′-5′ directionality and (b) the 3′-part of an Intron with 3′-5′ directionality further comprising a functional sequence region F2 downstream of the splice acceptor site.

There is also provided a method herein wherein said excised nucleic acid sequence, as disclosed previously herein, comprises;
(a) a nucleic acid sequence encoding a first selection marker;
(b) a promotor nucleic acid sequence P1; and/or
(c) an expression cassette encoding a second selection marker.

The above-mentioned design of the excised nucleic acid sequence provides for the selection of cells not having randomly integrated a donor vector in other locations than the pre-defined genomic location based on the expression of a second selection marker (SM2). This means that using such a design, the expression of SM2 would be positive following action of said second DNA enzyme only for a cell having integrated the Donor vector outside the pre-defined genomic location. Accordingly, the second round of selection can use a negative selection step based on the expression of SM2 to remove cells having integrated a donor vector outside the pre-defined genomic location. The removal of the expression cassette encoding the second selection marker is also an improvement to the method as that will save energy for the cell which may instead be used for producing the protein of interest.

A first selection marker may be selected from the groups of (i) fluorescent proteins and (ii) heterologous cell surface markers, in addition to what has been mentioned elsewhere herein. The use of a fluorescent protein or a cell surface marker as a selection marker provides particular advantages as the selection can be performed using fast and direct isolation methods (based on i.e. FACS or MACS) as soon as the concentration of the first selection marker has increased above a certain limit (allowing detection of fluorescence above background in FACS and allowing efficient binding to magnetic beads in MACS). This is in contrast to selection markers based on metabolic enzymes or antibiotic resistance genes needing a prolonged and indirect isolation strategy based on cells with an activated selection marker slowly out-growing cells lacking active selection marker.

The first DNA enzyme may be provided in the form of a plasmid, mRNA or a purified protein, optionally wherein said first DNA enzyme may be encoded by and expressed from said donor vector. The first DNA enzyme may also be expressed from an expression cassette encoding said first DNA enzyme which is present in the pre-defined genomic location of a cell of step i) of a method disclosed herein.

As previously mentioned herein, a donor vector of step ii) may further comprise an expression cassette encoding a second DNA enzyme, the expression of said second DNA enzyme being activated when said donor vector has been integrated into a pre-defined genomic location of a cell of step i) in a method disclosed herein.

The second DNA enzyme may also be provided in the form of a plasmid, mRNA or a purified protein.

A eukaryotic cell for use in a method presented herein may be selected from the group consisting of a yeast cell, a filamentous fungus cell, a plant cell, an insect cell or a mammalian cell. A mammalian cell may be a human, monkey, rodent or a mouse cell, but is not limited thereto. A eukaryotic cell is an isolated eukaryotic cell as previously mentioned herein. An isolated cell is a cell that has been isolated or removed from its natural environment.

A eukaryotic cell for use in a method presented herein may specifically be selected based on suitability for production of recombinant proteins in a bioreactor. A suitable cell can be selected from the group of CHO or HEK cell lines.

A eukaryotic cell for use in a method presented herein may specifically be selected based on similarity to a cell type present in a mammalian species such as humans.

A eukaryotc cell for use in a method presented herein may be selected from the group of cell lines capable of growing in suspension cultures.

Some key common theoretical benefits of the general SDI system forming part of the invention disclosed herein are:
(1) Enables selection steps minimizing the likelihood that an isolated cell differs from the desired outcome of having a single copy of an Integration Cassette (comprising a nucleic acid sequence variant) integrated at and only at the pre-defined genomic location. For optimization of nucleic acid sequence variants such as expression cassette designs based on transfection of a Donor vector mixture comprising a library of expression cassette designs this is a critical feature as there need to be a one to one correlation between a cellular phenotype and a single corresponding gene cassette design to ensure proper selection of nucleic acid sequence variants with the desired characteristics.
(2) Enables the option to retain only the sequence which contributes to the productivity of the cell line. No cellular resources are wasted on expression of selection marker protein and risks of unexpected interference between the nucleic acid sequence variant under investigation and selection marker cassettes are avoided. Presence of sequences of bacterial origin with a potential negative impact on long term expression stability of i.e. a GOI can be avoided, increasing the reliability of the method
(3) There is a flexibility in the choice of selection marker, as the selection marker is not part of the pre-defined genomic location sequence. Optimal selection markers can be selected based on the application.
(4) The desired integration event activates expression of the first selection marker allowing positive selection of cells with integration at the pre-defined genomic location with high specificity. Using selection markers such as fluorescent proteins or cell surface markers this enables very short time periods between transfection and selection of positive integrants (using e.g. FACS or MACS). Two to three days should be possible to obtain a result. This shortens the time needed to perform the method. In addition, early isolation of cells having undergone integration at the desired location from cells having undergone undesired integration events or no integration can have further benefits as it minimizes the risk of desired cells being outgrown (and possibly lost from the evaluation) by undesired cells.

Hence efficiency and performance of the method can be improved compared to methods lacking this feature.

Preferred implementations of the method utilizing serine recombinases such as PhiC31 or Bxb1 as a first DNA enzyme in combination with a single matching recombinase recognition sequence pair (i.e. attP/attB) further have the potential for superior integration efficiencies. Specifically so in combination with said promoter P1 functionally fused to the 5′-part of a split intron. PhiC31 or Bxb1 mediated recombination of their corresponding attP/attB pairs are irreversible reactions and hence in theory integration should be limited only by transfection efficiency and plasmid stability. This is in contrasts to Cre based integration or CRISPR/Cas9 based integration where competing non-productive reaction paths may exist and where, for cassette exchange reactions, efficiency will negatively correlate with the size of the integration cassette. The integration efficiency is a critical performance parameter as it directly impacts the number of nucleic acid sequence variants that can be evaluated simultaneously by the method. The integration efficiency for one preferred implementation of the method is exemplified in the experimental section, Example 1.

Hence, the present disclosure provides for a novel and improved way of an efficient and selective targeted integration of a multitude of nucleic acid sequence variants (for example encoding proteins of interest) into a population of isolated eukaryotic host cells. A population of isolated host cells having each selectively integrated a single copy of a donor vector comprising a nucleic acid sequence variant will present an excellent system for optimization of nucleic acid sequence variants with an impact on recombinant protein production or protein function and will find use in many different application areas.

Next, ways to perform the selection step based on a “defined phenotype” of an isolated eukaryotic cell having integrated a nucleic acid sequence variant at the pre-defined genomic location will be outlined together with associated applications. Accordingly, next it is described what is intended with the term “defined phenotype” herein. These descriptions are merely intended as examples and the present invention is not limited thereto.

In general the phenotypic selection step can be based on any characteristic of a cell that can be measured (using current or future analytical technologies) and the corresponding measurement result used to specifically target a cell for selection. The measurement and subsequent isolation of a cell targeted for selection can either be performed using (i) technologies capable of measurements and manipulations at the single cell level, such as FACS or (ii) by first isolating single cells in separate compartments allowing measurements using bulk analytical technologies and subsequent selection by discarding undesired compartments.

Alternatively the phenotypic selection step can be based on any characteristic of a cell that can be utilized to indirectly select or enrich for a phenotype based on either (i) differential growth under defined growth conditions or (ii) differential binding of cells to a solid phase such as a magnetic bead with subsequent isolation of bound or non-bound cells using i.e. MACS.

As each individual cell has carefully been selected to harbour a single nucleic acid sequence variant, selection based on one or several phenotypic characteristics will probe whether a specific sequence variant has either a negative, a neutral or a positive impact on said characteristic(s). In either case, a specific nucleic acid sequence variant corresponds to a defined phenotype in the sense that a measured cellular characteristic of cells harbouring said specific variant will display a defined statistical distribution. Selecting cells with a measured characteristic above a defined value results in selection or enrichment of nucleic acid sequence variants with an impact on said characteristic. Nucleic acid sequence variants can be selected/enriched for using any defined measurement interval of a defined phenotypic characteristic.

Hence, a sequence optimized nucleic acid sequence according to the present invention should be understood as a sequence corresponding to a defined measurement interval of a selected phenotypic characteristic. Optimized nucleic acid sequences selected and isolated via isolation of a defined cellular phenotype can be identified by sequencing the genomic DNA at the pre-defined genomic location. If genomic DNA is extracted from cloned cells (a population originating from a single cell), standard Sanger sequencing can be used. If genomic DNA is extracted from a diverse population of cells, parallel sequencing methods [20] can be used.

One application of the method is to probe nucleic acid sequence variants in a donor vector for their impact on the expression of a recombinant protein with a defined amino acid sequence. This has value for the commercial production of recombinant proteins such as therapeutic proteins, proteins used as standards or reagents in diagnostic tests or proteins used as reagents in research applications. As previously eluted to, many different functional sequence components have been shown to impact obtainable protein levels at different stages of expression [14-19].

Sequences impacting the transcription of a gene of interest (GOI) includes the choice of promotor (core promotor sequence including the start transcription site sequence, proximal promotor sequence and transcriptional enhancer sequences including their spacing to other promotor components) and the presence (or absence) of sequences impacting local DNA structure in the genome (chromatin modifying elements, chromatin insulators, matrix/scaffold attachment regions, etc.). Besides being impacted by the level of transcription, cytosolic mRNA levels are also impacted by the efficiency of nuclear export (impacted by presence, position and choice of intron sequences) and their cytosolic degradation rate (impacted by choice of 3′-UTR). Translation initiation is impacted by choice of 5′-UTR and 3′-UTR sequences and secretion by choice of signal peptide amino acid sequence. Besides these non-coding sequence elements, the nucleic acid sequence (synonymous coding sequence choice) used to encode the protein of interest (POI) has been shown to have a significant impact on many stages of expression including the folding of the polypeptide chain through a process referred to as co-translational folding [19].

For multichain proteins such as antibodies, the ratio of the different chains is critical to the observed secretion of functional protein. With the advances in nucleic acid synthesis technologies allowing control of nucleic acid sequences down to the base pair level the nucleic acid sequence design space is vast. Hence, large opportunities for improved expression, and accordingly improved manufacturing economy, can be gained by optimization of nucleic acid sequence variants present in a donor vector for a given recombinant protein.

In this application, said defined phenotype can for example be defined by a measurement of the expression of the protein of interest (i.e. the recombinant protein) falling into a specified range. One way of performing the phenotypic selection step is to perform single cell cloning (using for example FACS or limited dilution) from a population of cells generated by the SDI part of the disclosed method and measure the bulk titer after a defined period of culture. Clones giving a titer within the specified range are selected thereby selecting and isolating corresponding optimized nucleic acid sequence variants. In this implementation the diversity of nucleic acid sequence variants that can be investigated is limited by the number of clones that can be cultured and assessed in parallel. Using traditional parallel culture formats, such as static culture in micro titer plates, the number of clones is practically limited to around 10 000 (100 96-well plates). To ensure that biological and technical variation is covered this means that nucleic acid sequence variant libraries with a diversity of a few hundred can be efficiently examined. The library diversity can be slightly raised by using novel parallel culture and assessment technologies such as droplet manipulation technologies [21] or other microsystem-based technologies [22, 23].

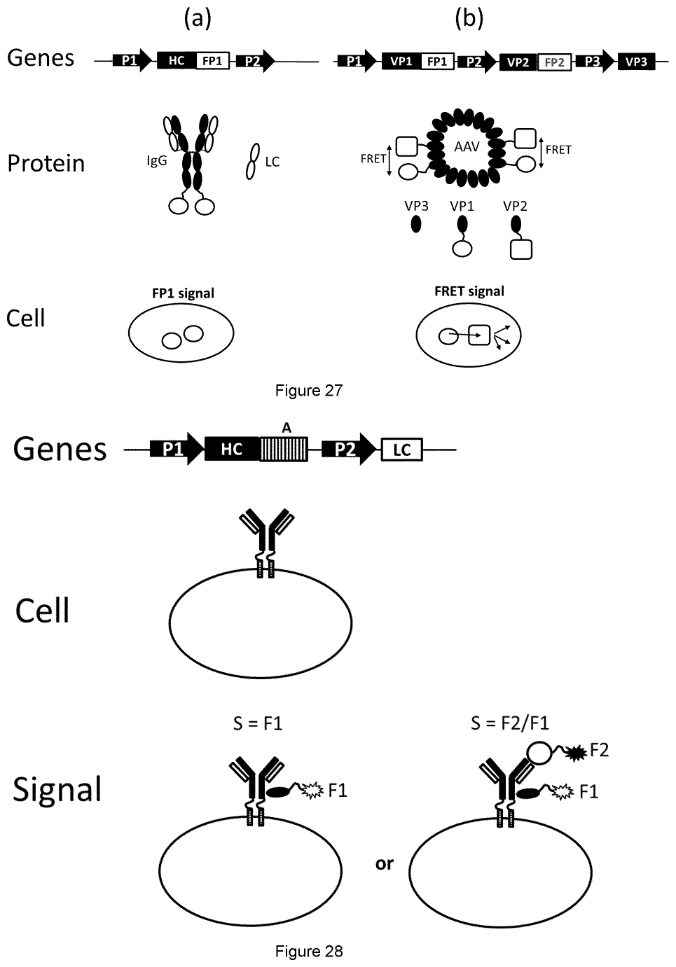

However, to significantly increase accessible library diversity, technologies capable of single cell measurements (such as FACS) are preferred. To enable an assessment of expression of a protein of interest by FACS, the protein of interest can be genetically fused to a fluorescent protein at the c- or n-terminal on one of its polypeptide chains, see FIG. 26 and FIG. 27a. To normalize for technical and biological noise an additional gene coding for a second fluorescent protein (with corresponding gene cassette kept constant for all Donor Vector variants in said plurality of donor vectors) can be utilized and the selection based on the ratio of the two fluorescent protein emissions, see FIG. 26b. For proteins of interest comprising multiple polypeptides, such as viral particles or bi-specific antibodies, genetic fusion of two different fluorescent proteins to two different polypeptide chains can be implemented to enable an assembly specific expression signal based on FRET activation between the two fused polypeptides when assembled in close proximity (See FIG. 27b). Other means to generate a measure of POI expression amendable to FACS analysis is to ensure tethering of the POI at the cell surface followed by binding of the POI to fluorescent detection reagents before running a FACS sort, see FIG. 28. This can be achieved through the fusion of the POI with a membrane anchor domain [13, 30]. Optionally, the presence of the membrane anchor domain on the POI can be controlled through the use of i.e. a leaky translational stop codon [24]. Cells giving fluorescence signals within a defined range can be isolated in one or optionally multiple successive FACS sorting steps (optionally using different fluorescence ranges). Besides increasing the obtainable diversity, methods such as FACS also enables cells to be cultured using culture formats and conditions relevant to large-scale production before assessment and sorting.

The defined phenotype can also be coupled to measurements of other cellular characteristics such pre-apoptotic stress signaling or specific stress signaling status such as ER stress, amino acid starvation or hypoxia.

Single-cell read-out of different cellular characteristics can potentially be enabled through the introduction of engineered and genetically encoded sensing and reporting circuits into a isolated eukaryotic host cell used in the present disclosure. See for example [25].

Using the disclosed method to identify sequence variants corresponding to a defined phenotype, nucleic acid sequence variants can be directly optimized for improving the expression of a given recombinant protein of interest. Data on the performance of different nucleic acid sequence variants obtained by the method can also be used to infer design rules that can be used to select donor vector designs for other related or un-related recombinant proteins. Such design rules can for example be based on machine learning or other artificial intelligence techniques. As outlined in FIG. 1, the method can also be used to directly isolate a cell for downstream applications such as production of a protein of interest. Finally, the method can be utilized to isolate optimized nucleic acid sequence variants for direct downstream applications such as incorporation in donor vectors for Cell Line Development (CLD).

Another application of the method is to evaluate different recombinant proteins (i.e. differences in amino acid sequence) for their expression in a given type of eukaryotic cell. Implementation of the method for this application can utilize any of the example designs described previously for optimization of donor vector sequences. Evaluation of different amino acid sequences can be done to screen early therapeutic protein candidates for their manufacturability in the final intended production host or to improve the expression, manufacturability or developability of a specific therapeutic protein candidate through the introduction of amino acid sequence diversity at positions outside its functional surface [27]. The method can also be used to improve the functionality of naïve libraries for discovery of new therapeutic protein candidates based on target binding (i.e. antibody discovery using naïve antibody libraries). A diversity of different scaffold designs can be screened for their impact on expression both to select a scaffold to use for a new library design and to infer design rules for library design and construction for a given scaffold.

Besides the amino acid sequence of the POI (including potential signal peptides), the nucleic acid sequence used to encode the POI (i.e. the GOI) and the non-coding sequences used in the donor vector, expression is also impacted by the cellular machinery present in the isolated eukaryotic cell. A natural diversity in the cellular machinery between individual cells is typically present and is exploited by clone screening campaigns to find the best possible production host for a given protein, introduced using a given donor vector.

However, to go beyond natural diversity, cell line engineering approaches can be utilized [26]. A challenge lies in the complexity of the cell and that current knowledge implies that the effect of specific engineering strategies can often be protein and even clone specific.

Hence, yet another application of the method disclosed herein enables improved means of performing protein and clone specific cell engineering. Cell engineering can for example be based on the introduction of different effector genes such as (i) expression cassettes coding for naturally occurring proteins (for over-expressions) or proteins not present in the used cell (introduction of new functionality), (2) introduction of genes encoding natural or engineered transcription factors for the control of endogenous gene expression or (3) introduction of genes encoding natural or engineered regulatory RNA such as miRNA or lncRNA for the manipulation of cellular pathways at a global or local level.

Based on the knowledge of the bottlenecks related to the clone and the protein produced, a library of different nucleic acid construct variants (each containing one or several effector genes) can be designed and constructed as a library of donor vectors. An isolated eukaryotic cell already stably expressing a given recombinant protein of interest and comprising a pre-defined genomic location according to the present invention (see Example 3 in the experimental section) is used to isolate a population of cells wherein each cell carries one nucleic acid construct variant comprising one defined set of effector gene(s) at the pre-defined genomic location. The phenotypic selection step and sequence identification can be performed using the same basic techniques as described previously.

In a different application the method can be used to select a recombinant protein sequence with a desired functionality. A typical example is the screening of a library of amino acid sequence variants (encoded by nucleic acid sequence variants) for their binding characteristics (i.e. function) to a specific target molecule or structure (i.e. such as in antibody discovery).

A typical library is constructed based on the introduction of sequence diversity at key positions of an otherwise conserved protein sequence often referred to as a protein scaffold. Examples of protein scaffolds includes the IgG1 scaffold, the nanobody scaffold and the Z-domain scaffold [27-29]. To enable screening of variants expressed from a single cell, protein scaffold variants are genetically fused to a membrane anchor domain to enable display on their surfaces and screening by a method referred to as mammalian display [13, 30].

The present invention offers improved means to perform mammalian display based on the features of the disclosed SDI method discussed previously. In the first part of the method, a library of amino acid sequence variants (all fused to a membrane anchor domain) comprised by a plurality of donor vectors of the present disclosure is used to generate a population of isolated eukaryotic cells highly enriched for cells carrying a single amino acid sequence variant from said library at and only at said pre-defined genomic location (and hence a single amino acid variant displayed on the surface).

In this application, said defined phenotype can for example be defined by a measurement of the binding of said target structure to a surface displayed amino acid variant falling into a specified range. In a preferred implementation the target structure (for example a protein) is labelled with a fluorophore and incubated together with the population of isolated eukaryotic cells carrying amino acid sequence variants on their surfaces, see FIG. 28. Following incubation and washing of the cells, FACS is used to record amino acid sequence variant to target binding by fluorescence. Cells giving a fluorescence reading within a specified range is isolated. Typically, iterative FACS isolation steps following incubations with reduced concentrations of labelled target are used to enrich for high affinity binders. Optionally, the target binding signal can be normalized by the amount of amino acid sequence variant displayed on the cell. This can be achieved by incubation with a fluorescently labelled reagent (with fluorescence non-overlapping with target fluorescence) binding to a conserved epitope present in all displayed amino acid sequence variants (i.e. the FC part of the IgG1 scaffold), see FIG. 28. In another preferred implementation, the target (i.e. a protein) is presented on the surface of an eukaryotic cell expressing a fluorescent protein in the cytosol.

Another example of application of the method to select a recombinant protein sequence with a desired functionality is the optimization of the product quality of a therapeutic protein. Here, selection is based on characteristics such as the glycosylation profile and the conformation (protein fold) of the recombinant protein. By display of a recombinant protein variant on the surface of a cell, glycosylation and conformation can be assessed by incubation with fluorescence labelled reagents such as glycoform specific affinity binders and/or conformation sensitive affinity binders (such as a target for an antibody) followed by measurement and isolation by FACS. Optionally, reagent binding signals can be normalized by the amount of recombinant protein variant displayed on the cell. See FIG. 28.

Other examples of application of the method to select a recombinant protein with a desired function includes: (i) Discovery or optimization of fluorescent proteins using direct isolation of desired variants by FACS, (ii) Discovery or optimization of enzymes for which enzymatic activity infers a detectable difference in phenotype, such as enzymes giving antibiotic resistance, (iii) Discovery or optimization of recombinases for integration of a donor vector into a pre-defined genomic location through contacting a population of isolated eukaryotic cells carrying a recombinase variant at and only at said pre-defined genomic location and a second pre-defined genomic location according to the present invention with a donor vector and isolating positive integrants via a positive FACS sort of cells having activated said first selection marker or (iv) Development and optimization of genetically encoded signaling and/or logics circuits [25] by integration of a multitude of candidate circuit designs generating a measurable cellular output followed by selection of variants with desired performance.

Accordingly, in a first aspect, the present invention relates to a method for the selection of a sequence optimized nucleic acid sequence from a plurality of nucleic acid sequence variants, wherein said sequence optimized nucleic acid sequence corresponds to an isolated eukaryotic cell with a defined phenotype, said method comprising:
i) Providing a population of isolated eukaryotic cells, each cell comprising a pre-defined genomic location, which pre-defined genomic location comprises:
a. a nucleic acid sequence I1 comprising a recognition site for a first DNA enzyme;
b. a nucleic acid sequence E1 comprising a recognition site for a second DNA enzyme; and
c. a promotor nucleic acid sequence;
ii) Providing a plurality of donor vectors, each donor vector comprising:
a. a nucleic acid sequence I2;
b. a nucleic acid sequence E2 comprising a recognition site for said second DNA enzyme;
c. a nucleic acid sequence encoding a first selection marker; and
d. a nucleic acid sequence region comprising a nucleic acid sequence variant,
iii) Contacting the plurality of donor vectors with the population of cells in the presence of a first DNA enzyme, wherein the presence of the first DNA enzyme enables recombination between the nucleic acid sequence I2 of a donor vector and the nucleic acid sequence I1 present in the pre-defined genomic location of a cell;
iv) Selecting and isolating a cell having a donor vector integrated at the pre-defined genomic location by detecting the expression of the first selection marker in the cell, wherein the expression of the first selection marker is activated by the promotor nucleic acid sequence at the pre-defined genomic location; and
v) Selecting and isolating a cell with a defined phenotype from the cells of step iv), thereby selecting and isolating a sequence optimized nucleic acid sequence from said nucleic acid sequence variants, said sequence optimized nucleic acid sequence corresponding to said defined phenotype.

The first selection marker may also be abbreviated and referred to as “SM1” herein.

The second selection marker may also be abbreviated and referred to as “SM2” herein.

Herein, said sequence optimized nucleic acid sequence corresponds to a eukaryotic cell with a “defined phenotype”, a “defined phenotype” meaning that for analysis and selection purposes of said optimized sequence variant, a cell having a particular physical characteristic or behavior as previously defined and discussed herein will be selected. As also previously defined herein, the phenotypic selection step can in general be based on any characteristic of a cell that can be measured (using current or future analytical technologies) and the corresponding measurement result used to specifically target a cell for selection.

The use of a plurality of donor vectors comprising different nucleic acid variants and a population of host cells means that it is possible to evaluate more than one nucleic acid sequence in the same run. Naturally, not every single cell will integrate a different nucleic acid sequence variant, however, the method allows for a broad multiplex approach making it possible to evaluate a significant number of nucleic acid sequence variants simultaneously.

There is also provided a method herein, wherein each of the plurality of donor vectors of step ii) further comprises:
e. an expression cassette encoding a second selection marker; and
wherein said method further comprises the steps of:
vi) Excising a nucleic acid sequence flanked by the nucleic acid sequences E1 and E2 from a cell of step iii) or selected and isolated in step iv) or step v) in the presence of a second DNA enzyme, wherein the presence of the second DNA enzyme enables recombination between the nucleic acid sequences E1 and E2, wherein the presence of an expression cassette encoding a second selection marker that is not flanked by the nucleic acid sequences E1 and E2 in a cell is indicative of a stable integration of a donor vector at a genomic location outside the pre-defined genomic location of said cell; and
vii) Selecting and isolating a cell from step vi) lacking an expression cassette encoding a second selection marker.

There is also provided a method herein, further comprising obtaining nucleic acid sequence information from the isolated cell of step iv), v) or vii) by sequencing of the sequence optimized nucleic acid sequence present at the pre-defined genomic location of the isolated cell. Sequencing of an optimized nucleic acid sequence present at a pre-defined genomic location can be performed by sequencing methods well-known to a person skilled in the art [20]. Sequencing is performed to identify the sequence variant that has functioned most efficiently in a SDI system according to the present disclosure in comparison to variants thereof that may not have worked as efficiently.

As previously mentioned herein, a nucleic acid sequence variant of the plurality of donor vectors may constitute a variant of a promotor, a variant of an intron, a variant of a transcription regulatory sequence, a variant of a DNA structure regulatory sequence, a variant of a 5′ untranslated region, a variant of a 3′ untranslated region, a variant of an internal ribosome entry site, a variant of a gene of interest, a variant of a nucleic acid sequence encoding a signal peptide and/or any combination of such variant. One can also envisage other examples of nucleic acid sequence variant categories that may be evaluated in the context of the present disclosure to identify an optimized sequence variant out of a plurality of nucleic acid sequence variants of a particular sequence category.

There is also provided a method wherein the selection of a sequence optimized nucleic acid sequence by the selection of a cell with a defined phenotype in step v) comprises selection based on one or more of the following phenotypic characteristics of said cell:
(i) The presence or expression level of an endogenous biomolecule in said cell;
(ii) The expression level of a recombinant protein of interest by said cell;
(iii) The growth rate of said cell; and/or
(iv) The functionality of a recombinant protein of interest encoded by said nucleic acid sequence region in said cell.

Said recombinant protein of interest may be a recombinant fusion protein, such as a protein fused to a membrane anchor domain for localization at said cell surface and/or is a protein fused to a fluorescent protein or a fluorescent protein domain. Herein, said endogenous biomolecule may constitute a protein, an mRNA, an miRNA (micro RNA), an lncRNA (Long non-coding RNA) or a metabolite, but is not limited thereto.

As previously mentioned herein, the selection of a sequence optimized nucleic acid sequence by the selection of a cell with a defined phenotype in step v) of a method presented herein may comprise selection based on the functionality of a recombinant protein of interest. Said functionality of a recombinant protein of interest may be measured and determined based on an interaction between said recombinant protein of interest when localized and expressed at the cell surface of said cell, and a target structure, such as a small molecule, a DNA molecule, an RNA molecule, a protein, a protein complex such as a virus particle, an exosome or a cell, optionally wherein said target structure is tagged with a fluorescent moiety. Which fluorescent moiety to use in the context of a method according to the present invention will be apparent to a person skilled in the art.

More specifically, said recombinant protein of interest may also be an affinity protein candidate wherein the level of expression is determined by display of said affinity protein candidate on the cell surface of said cell. Said affinity protein candidate may be a single chain polypeptide fused to a membrane anchor domain, optionally wherein said single chain polypeptide may be selected from the group consisting of a Z-scaffold protein, a Nanobody scaffold protein, a single chain fragment variable (scfv) scaffold protein, a Fynomer scaffold protein, a DARPin scaffold protein and/or an adnectin scaffold protein [28, 29].

Said affinity protein candidate may also comprise two or more polypeptide chains, such as an antibody, wherein a nucleic acid sequence variant corresponding to said affinity protein candidate encodes an affinity protein candidate variant, such as an antibody variant, and wherein one of said two or more polypeptides, e.g. of said antibody variant is fused to a membrane anchor domain [13, 30].

There is also provided a method further comprising determining the binding specificity, selectivity, affinity and/or functionality of said affinity protein candidate, by providing to said affinity protein candidate a specific target component, optionally labelled with a fluorescent marker, to which the affinity protein candidate is exposed and thereafter detecting binding of said affinity protein candidate to said specific target component.

Said target component may be selected from a small molecule, a DNA molecule, an RNA molecule, a protein, a protein complex, such as a virus particle, an exosome or a cell, optionally wherein said target structure is tagged with a fluorescent moiety.

Said affinity protein candidate may be any protein capable of binding or having affinity to said target component.

As previously mentioned herein, the selection of a sequence optimized nucleic acid sequence by the selection of a cell with a defined phenotype in step v) of a method presented herein may comprise selection based on the expression level or functionality of a recombinant protein of interest and/or said presence or level of an endogenous biomolecule. Such a presence or level may be measured at the level of a single cell, such as by using Flow Cytometry.

There is also provided a method wherein said nucleic acid sequence region of a donor vector comprises a nucleic acid sequence variant for expression of one or several recombinant proteins of interest from said donor vector, wherein said plurality of donor vectors comprises different nucleic acid sequence variants encoding different amino acid sequence variants of said one or several recombinant proteins of interest.

There is also provided a method, wherein said nucleic acid sequence region of a donor vector comprises a nucleic acid sequence variant for expression of a recombinant protein of interest from said donor vector, wherein nucleic acid sequence variants present in a plurality of donor vectors comprises nucleic acid sequence variants encoding essentially identical amino acid sequence variants of said recombinant protein of interest. Such a method is envisaged to identify an optimal nucleic acid sequence of interest to encode a protein of interest.

There is also provided a method comprising a plurality of donor vectors comprising a nucleic acid sequence region comprising essentially identical nucleic acid sequences to encode essentially identical recombinant proteins of interest but wherein said nucleic acid sequence region comprises different nucleic acid sequence variants of donor vector components, such as nucleic acid sequence variants of promoter or enhancer nucleic acid sequences of said donor vector. Such a method is envisaged to identify an optimal nucleic acid sequence variant to drive expression and secretion of said recombinant protein of interest.

Accordingly, such a method identifies sequence optimized nucleic acid donor vector components for use in recombinant protein expression systems based on a eukaryotic cell system. Accordingly, in this regard there is also provided a sequence optimized nucleic acid sequence selected by a method as disclosed herein.

There is also provided the use of such a sequence optimized nucleic acid sequence, for producing a recombinant protein.

There is also provided the use of a sequence optimized nucleic acid sequence selected by a method as disclosed herein, for designing further sequence optimized nucleic acid sequences. This means that the information of the sequence identity of the sequence optimized nucleic acid sequence selected by the method as disclosed herein is used to produce additional optimized nucleic acid sequence variants. Accordingly, this information is used for design purposes for the design of additional variants of a selected nucleic acid sequence.

In a further aspect of the present disclosure, there is provided an isolated eukaryotic cell with a defined phenotype corresponding to a sequence optimized nucleic acid sequence obtainable by a method as disclosed herein.

The present disclosure will now be illustrated by the following experimental section without intending to be limited thereto as it merely illustrates different ways of performing the invention.

### EXPERIMENTAL SECTION

### Abbreviations

LP=Landing Pad
LP1P1=Landing Pad 1 comprising attP1
LP2P2=Landing Pad 2 comprising attP2 and a split intron
CHO=Chinese Hamster Ovary
FC-eGFP=Enhanced Green Fluorescent Protein fused to an FC from IgG1
TagBFP2=Blue Fluorescent Protein variant
TagRFP-T=Red Fluorescent Protein Variant
G418=Also known as geneticin, a broad-spectrum antibiotic that will select mammalian cells expressing the neomycin resistance gene (NeoR).

### Example 1: Efficiency of phiC31 Recombinase-Mediated Integration and Selection Marker Activation

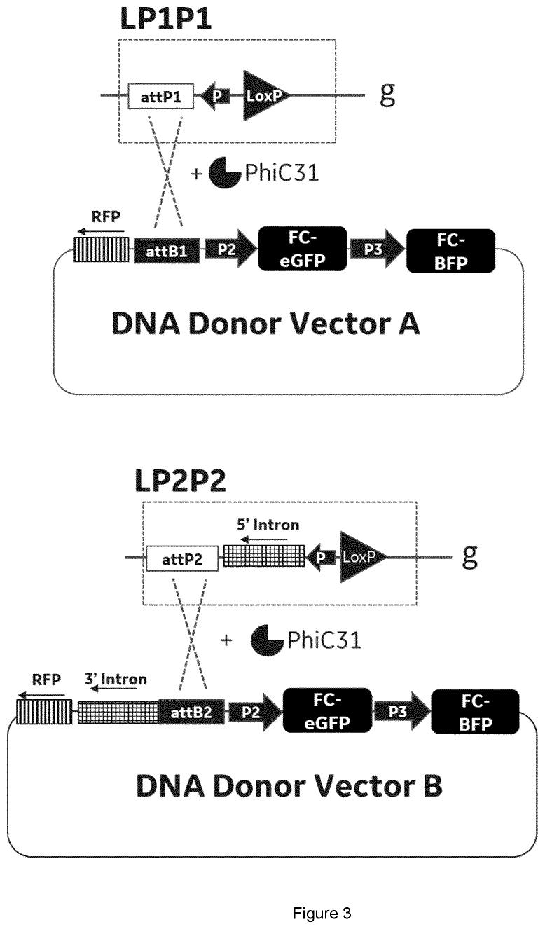

To investigate the efficiency of integration, HyClone CHO LP cells and non-LP HyClone CHO control cells were transfected with a combination of PhiC31 recombinase expression plasmid and either of Donor vector A or B (FIG. 3.). The Donor vectors contain expression cassettes for FC-eGFP and FC-TagBFP2 and a promoter-less TagRFP-T gene positioned so that it activates upon integration at the LP in LP cells.

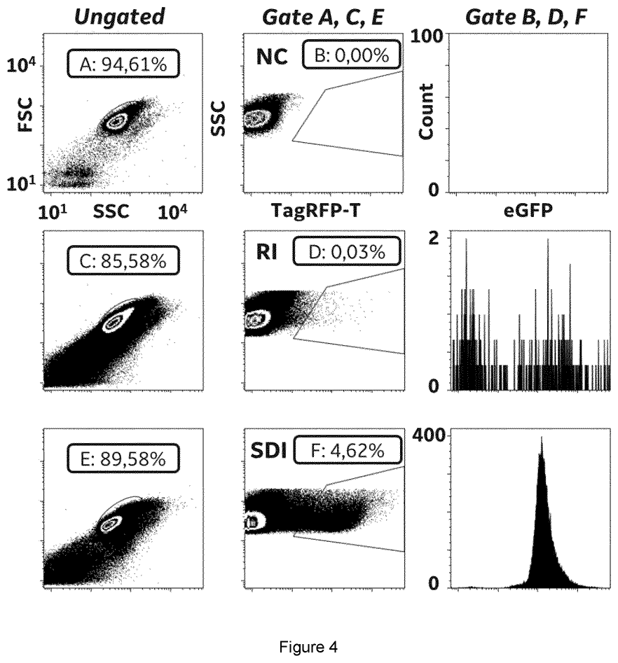

Two HyClone-CHO LP variants and matching Donor vectors were investigated, see FIG. 3. In HyClone-CHO LP1P1, the promotor in the LP is positioned directly downstream of attP1. In Donor vector A, the TagRFP-T gene is positioned directly upstream of attB1. In HyClone-CHO LP2P2, the 5′-part of a split intron is positioned between attP2 and the downstream promotor at the LP. In Donor vector B, the 3′-part of a split intron is positioned between attB2 and the upstream TagRFP-T gene. Efficiency of integration was evaluated by flow cytometry 7 days post transfection by measuring the percentage of cells displaying RFP signal above background (defined in comparison to non-transfected control, see FIG. 4).

In FIG. 4 an example of the flow cytometry data generated is illustrated for HyClone CHO LP2P2 in comparison to controls. The full set of results are summarized in Table 1. For LP1P1 only a non-transfected control was used whereas for LP2P2 both a random Integration control (RI Control, Donor vector only) and a pseudo-att integration control (Donor vector+PhiC31 in a CHO cell line lacking the LP) were performed. According to the data both LP variants are functional but the LP2P2 variant utilizing a split intron design gives superior integration efficiency.

TABLE 1
Summary of results obtained from Control experiments
and SDI integration efficiency evaluations for
both HyClone CHO LP1P1 and HyClone CHO LP2P2.

| Experiment | Cell Line | Plasmids | TagRFP |
|---|---|---|---|
| Non-transfected | HyClone CHO | — | 0% |
| Control | | | |
| LP1P1 SDI | HyClone CHO | PhiC31 + Donor A | 0.38% |
| | LP1P1 | | |
| RI Control | HyClone CHO | Donor B | 0.03% |
| Pseudo-att Control | HyClone CHO | PhiC31 + Donor B | 0.19% |
| LP2P2 SDI | HyClone CHO | PhiC31 + Donor B | 4.62% |
| | LP2P2 | | |

### Example 2: Efficiency of Cre Recombinase-Mediated Excision of Vector Backbone at the Integration Site

HyClone CHO LP1P1 cells were transfected using a PhiC31 expression plasmid and a Donor Vector containing expression cassettes for FC-eGFP and FC-TagBFP2 and a promoter-less TagRFP-T gene positioned so that it activates upon integration at the LP in LP cells (FIG. 5). Cells having integrated the Donor Vector at the Landing Pad (LP) were enriched by several FACS sorting steps gating for Tag-RFP-T signal above background and a balanced expression of both FC-eGFP and FC-TagBFP2. The resulting sorted and expanded pool of cells was then transfected a second time using either (a) a Cre recombinase expression plasmid, (b) a synthetic mRNA encoding Cre or (c) a mock transfection solution lacking any Cre recombinase encoding nucleic acid molecule. Seven days post the second transfection all cell populations were analyzed by flow cytometry to evaluate the efficiency of excision of the region flanked by two IoxP sites.

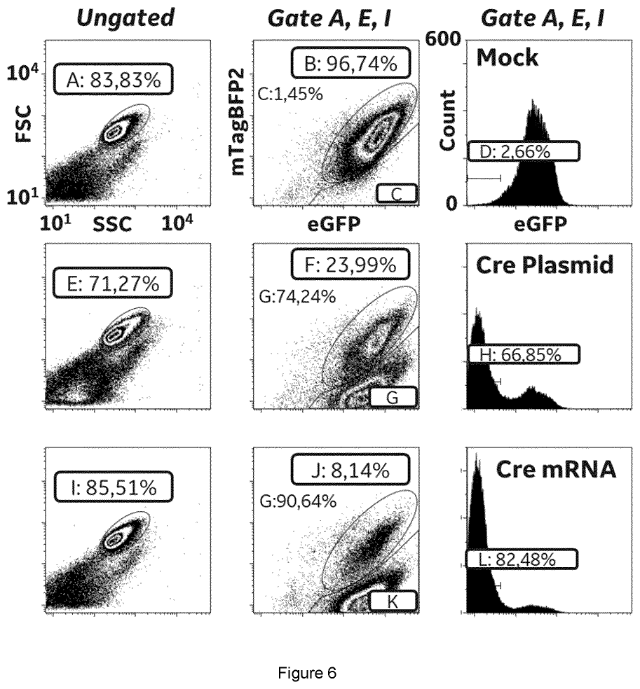

Plots from the flow cytometry analysis following the Cre recombinase transfection can be seen in FIG. 6. The data shows an increase of cells that do not express FC-TagBFP2 for the two Cre recombinase treated pools as compared with the mock control. This in turn clearly indicates correct integration of the donor vector at the LP so that FC-TagBFP2 are flanked by two IoxP sites which the Cre recombinase enzyme can act on. According to the data, the excision reaction catalyzed by Cre recombinase is highly effective with a yield up to at least 80%.

### Example 3: Repeated Integration at the Same Genomic Location by the Using of Orthogonal attP/attB Pairs

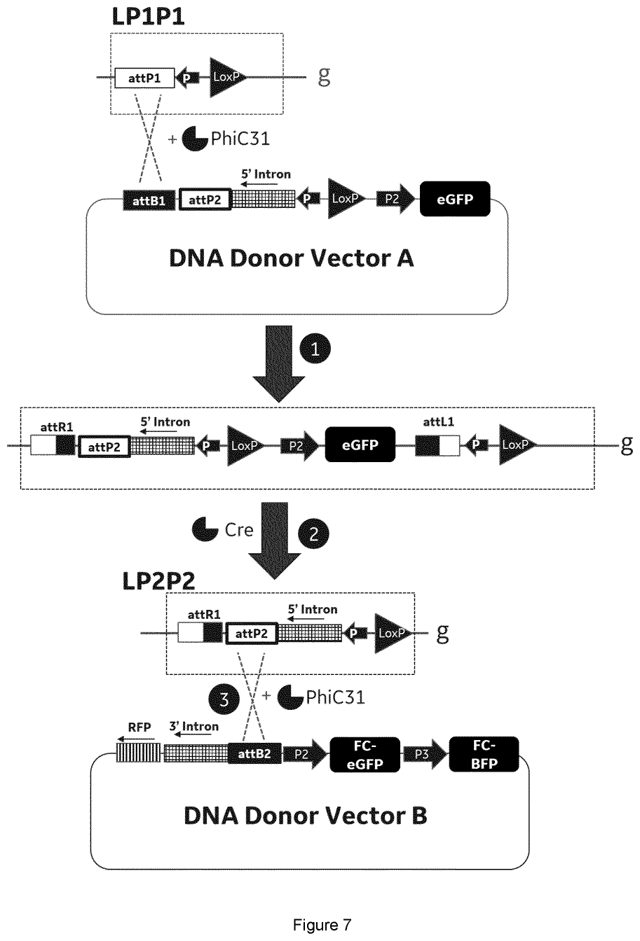

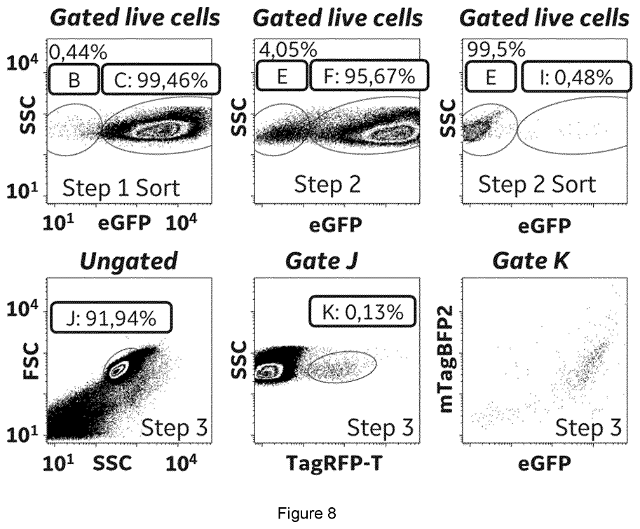

HyClone CHO LP1P1 cells were transfected using a PhiC31 expression plasmid and a Donor Vector containing an attB1 sequence followed by an attP2 sequence, the 5′-part of a split intron, a promotor, a IoxP sequence and an expression cassette for eGFP (FIG. 7, Donor Vector A). At day 7 post transfection, eGFP positive cells were sorted by FACS followed by expansion of cells. A second more stringent sort of eGFP positive cells were then performed. Cells expanded after the second eGFP positive sort (FIG. 8, Step 1 Sort) were transfected using a synthetic mRNA encoding Cre recombinase. Seven days post the Cre recombinase transfection, eGFP negative cells were sorted using FACS. Following expansion, the eGFP negative cell pool was analyzed by flow cytometry (FIG. 8, Step 2 Sort). Data from the sorting and analysis steps are shown in FIG. 8, upper panel. During these steps the Landing Pad in the CHO genome is assumed to have been altered as indicated by steps (1) and (2) in FIG. 7.

To verify functionality of the altered Landing Pad, the eGFP negative pool obtained after the final sort (FIG. 8, Step 2 Sort) were transfected using DNA Donor Vector B (FIG. 7, step 3) and analyzed by flow cytometry 7 days post transfection (FIG. 8, lower panel). Data indicates functionality of the new Landing pad. Finally, cells from the eGFP negative pool were cloned using single cell sorting by FACS and the Landing Pad region of their genomes amplified by PCR and sequenced. Correct alteration of the Landing Pad was confirmed for multiple clones by sequencing (full coverage of new Landing Pad region) showing that the alteration outlined in FIG. 7 has been successfully achieved.

### Example 4: Generation of an SDI Cell Pool Using Two Consecutive Selection Steps

HyClone CHO LP2P2 cells (Clone generated according to Example 3) were transfected using a PhiC31 recombinase expression plasmid and a Donor Vector constructed according to FIG. 9.

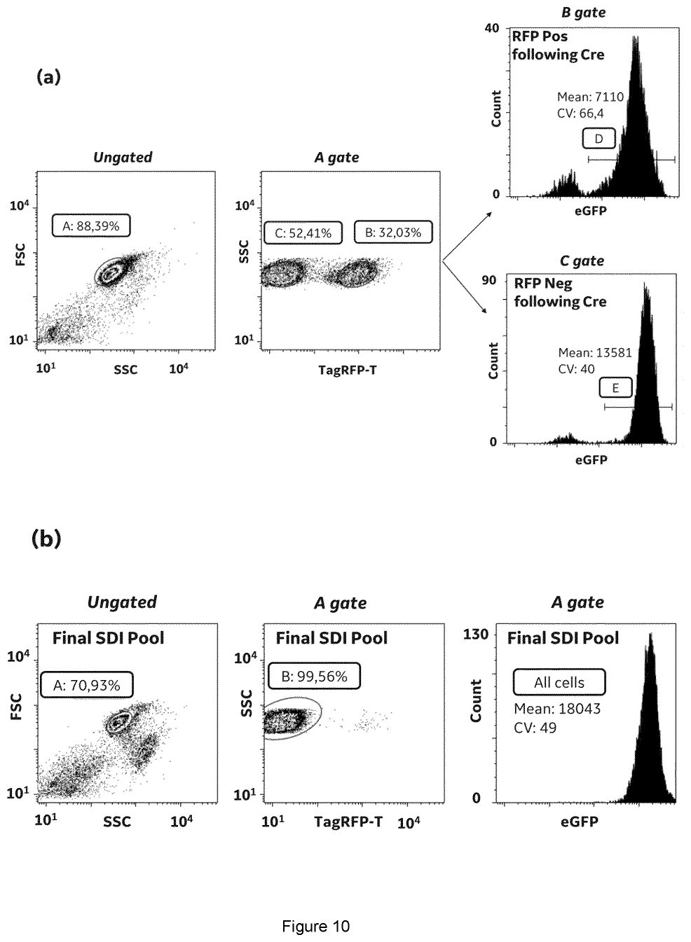

Starting at two days post transfection, cells were cultured in the presence of G418 to select for cells having integrated the Donor Vector at the Landing Pad and thereby activated the neomycin resistance gene (Neo^{R}). Following return to high viabilities (>98%) post G418 selection, the cells were sorted by FACS based on GFP/RFP double positive signal. Following expansion, the sorted cells were transfected using synthetic mRNA encoding Cre recombinase. At day 7 post Cre recombinase transfection, cells were FACS sorted by GFP-positive/RFP-negative signal (FIG. 10, gate E). The final SDI pool was analyzed by flow cytometry following expansion.

Data from FACS/Flow Cytometry can be seen in FIG. 10. The additional selection step following Cre recombinase transfection reduces the heterogeneity in the pool as indicated by the mean value and CV of the eGFP signal for TagRFP-T positive (incorrect integration) and TagRFP-T negative (correct integration) cells.

### Example 5: Evaluation of Combinations of Different 5′-UTRs and Signal Peptides for High Expression

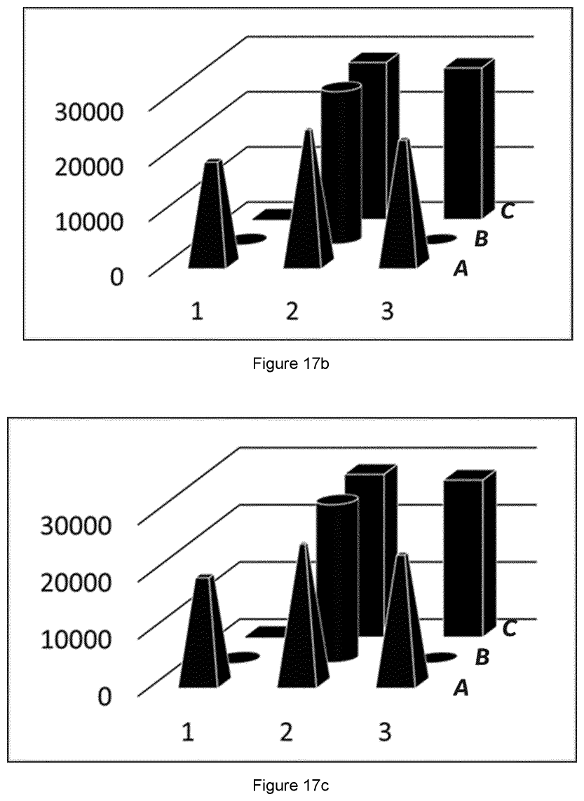

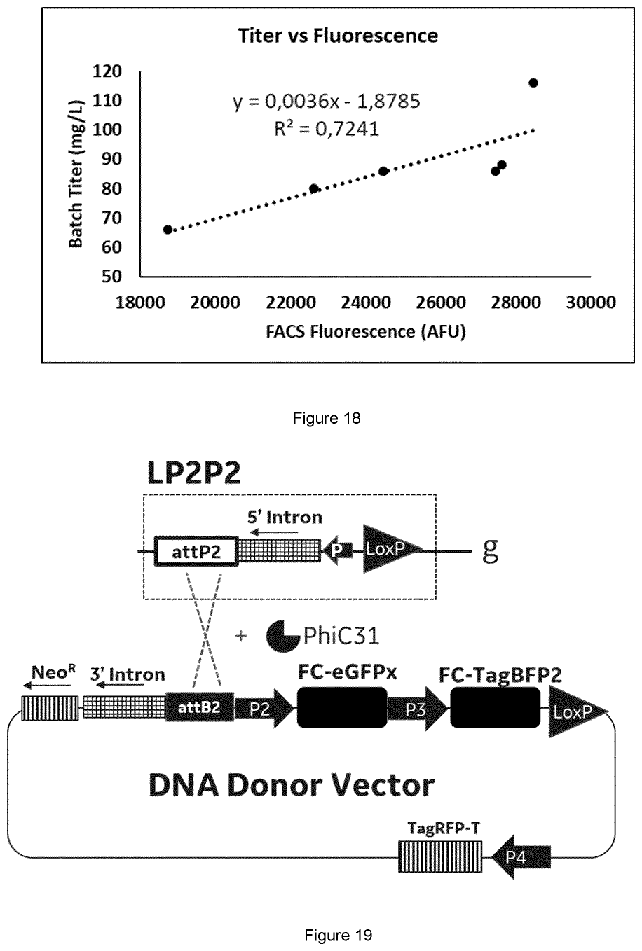

Nine different constructs were made by combining three different 5′-UTRs (A, B, C) and three different signal peptides (1, 2, 3) for expression of Fc-eGFP where the preferred combinations A1, B2 and C3 with known good expression from earlier experiments are in bold, see table 2. Each constructed donor plasmid was transfected together with PhiC31 plasmid to HyClone CHO LP2P2 cell line for site directed integration. After 2 days, the transient expression of eGFP and mTagBFP2 were analyzed with flow cytometry. The number (%) of cells with good expression of eGFP and mTagBFP2 can be seen in FIG. 17a and shows good values for preferred variants A1, B2 and C3. The neomycin selection marker was activated when donor vector was inserted in landing pad (LP) and therefore G418 was added after day 2 as selection pressure. When all cultures reached >98% viable cells again, mRNA of Cre enzyme was transfected to the cells to remove RFP and Neomycin expression cassettes. After one week, single cells were sorted for eGPF and mTagBFP2 positive and RFP negative signals for each sample, respectively. During G418 selection, cultures with constructs B1, B3 and C1 were lost. A batch culture for each remaining sample was performed and fluorescence (eGFP and mTagBFP2) was measured on cultures in log-phase (FIG. 17b, eGFP fluorescence) and FC titers were measured with CEDEX after culturing (FIG. 17c, mg/L). A ranking of the constructs can be seen in table 3. There is good correlation between the results from fluorescent signal and FC based titer measurement, see FIG. 18. The result shows that UTR C and signal peptide 2 gave the better expression, and, that the combination of them also generated the highest expression.

TABLE 2
Overview of the UTRs and signal peptides (SP) for the
nine different constructs. Combinations with known high
performance based on previous data indicated in bold.

SP

| UTR | SP 1 | SP2 | SP3 |
|---|---|---|---|
| UTR A | A1 | A2 | A3 |
| UTR B | B1 | B2 | B2 |
| UTR C | C1 | C2 | C3 |

TABLE 3
Ranking of expression of Fc-eGFP by flow
cytometer (MFI) and CEDEX (titer)

| Construct | MFI of eGFP | Fc titer (mg/L) |
|---|---|---|
| C2 | 28458 | 116 |
| B2 | 27622 | 88 |
| C3 | 27458 | 86 |
| A2 | 24481 | 86 |
| A3 | 22625 | 80 |
| A1 | 18739 | 66 |

### Example 6: Evaluation of 5′-UTR Variants by Flow Cytometry Using Fc-eGFP as a Single Cell Qp Probe

To evaluate the ability to distinguish different expression levels of a relevant target gene at the single cell level, four different DNA donor vectors with varying gene constructs for a signal peptide containing Fc-eGFP were constructed (See FIG. 19 for general design of the donor vectors and Table 4 for a description of the variants). Three of the variants differ only in their 5′-UTR sequence whereas the Negative control vector had the full Fc-eGFP cassette including the promotor deleted. For all four vectors an identical signal peptide containing Fc-TagBFP2 gene cassette is present as an internal control.

TABLE 4
Description of DNA donor vector Variants.

| Variant | Description | Kozak sequence | uORF sequence |
|---|---|---|---|
| pGE0381 | Positive control | GCCGCCACC | — |
| pGE0462 | Strong negative attenuation. | ACC | ACCATGGGTTGA |
| | Three uORFs placed upstream | | (SEQ ID NO: 15) |
| | of short Kozak. | | |
| pGE0464 | Medium negative attenuation. | ACC | CAAATGGGTTGA |
| | One uORF placed upstream of | | (SEQ ID NO: 16) |
| | short Kozak. | | |
| pGE0490 | Negative control. FC-eGFP | — | — |
| | cassette including promotor | | |
| | and 3′-UTR deleted. | | |

HyClone CHO LP2P2 cells were transfected using a PhiC31 expression plasmid and either of the four DNA Donor Vectors. Individual transfections and selections were performed for all DNA Donor Vectors. Starting at two days post transfection, cells were cultured in the presence of G418 to select for cells having integrated the Donor Vector at the Landing Pad and thereby activated the neomycin resistance gene (Neo^{R}). Following return to high viability (>98%) post G418 selection the cells were analyzed by flow cytometry. For comparison of the different variants, the cell populations were gated in-silico according to the upper panel in

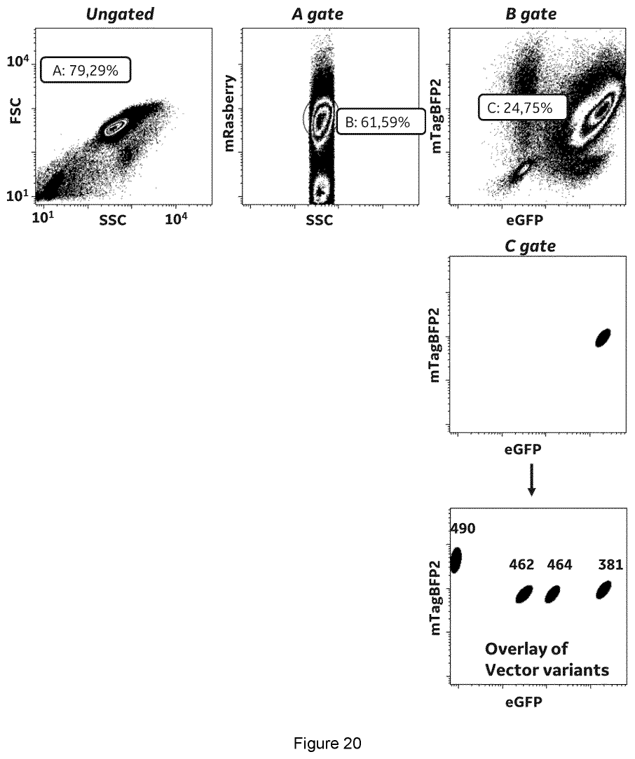

FIG. 20. From the total live cell population, the main cluster of RFP positive cells was selected and a plot of their TagBFP2/eGFP values displayed. The main cluster of TagBFP2/eGFP positive cells was gated out and used for comparison of the different variants. An overlay plot of these gated populations can be found in the lower panel of FIG. 20.

The data comparing the expression generated using the different variants shows a wide dynamic range of Fc-eGFP responses. Further, ranking based on Fc-eGFP response correlates with the expected performance of the different constructs, with the high efficiency positive control showing the highest response and the Negative control (absence of Fc-eGFP expression cassette) does not yield a eGFP response above background. Based on published data in the scientific literature [31] the 462 triple uORF attenuation sequence should yield a stronger negative attenuation of the downstream gene compared with the 464 single uORF attenuation sequence. For the three variants with intact Fc-eGFP cassettes the signal responses for TagBFP2 are similar adding confidence to the eGFP ranking. For the negative control the TagBFP2 response is elevated. This could potentially be due to the absence of the Fc-eGFP promotor.

### Example 7: Comparison of 3′-UTR Variants by Flow Cytometry and Titer Assessment Using Fc-eGFP as a Single Cell Qp Probe

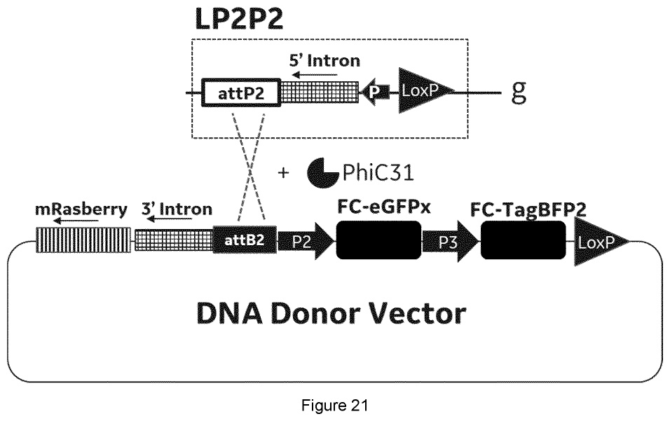

Eight (8) different DNA donor vectors carrying different 3′-UTR sequences in the Fc-eGFP cassette (see FIG. 21 for general design of the vectors) were constructed.

HyClone CHO LP2P2 cells were transfected using a PhiC31 recombinase expression plasmid and either of the eight DNA Donor Vectors. Individual transfections and selections were performed for all DNA Donor Vectors. Seven days post transfection, initial cell populations were FACS sorted based on mRasberry positive signal (corresponding to activation of the promotor-less gene at the Landing Pad). Following expansion, a second more stringent mRasberry positive FACS sort were performed to generate a pool of SDI cells for each DNA Donor Vector variant. Following expansion shake flask batch cultures were initiated with a starting cell density of 0.25×10⁶ cells/ml. Viable cell density was measured daily using a ViCell instrument. A sample from each culture were analyzed by flow cytometry at day three of culture. The titer in the growth medium was measured at day five of culture using a Biacore 8K+ instrument with an IgG1 standard for generation of a calibration curve.

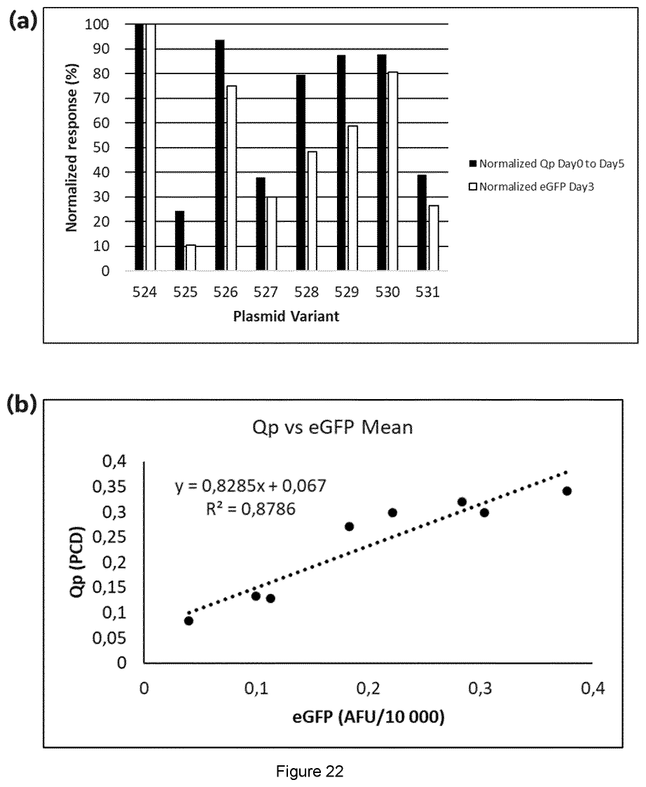

The Qp (day 0 to day 5) for each culture was calculated and compared to the average eGFP signal from the corresponding flow cytometry data (giving a proxy of Qp at day 3). The data can be found in FIG. 22.

As can be seen the Qp calculated from the titer and VCD measurements correlates with the flow cytometry data. By extension, this indicates that fusion of a fluorescent protein to a protein of interest can be used to select/enrich cells with high expression from a pool of cells collectively carrying a library of nucleic acid variants (such as 3′-UTR variants), thereby selecting/enriching for high performing nucleic acid variants.

### Example 8: Evaluation of a Small Library of Gluthamine Synthetase Variants for their Efficiency as Selection Markers

Three control DNA donor vectors and one DNA donor vector library with general design according to Example 4 were constructed. The vectors differ only in the sequence at codon 299 of the coding sequence for Glutamine synthetase (see FIG. 23 for details). The positive control, Arg with the codon CGA, is known to code for a high function enzyme (data not shown). The negative control, Gly with codon GGA, has been shown to work for MSX selection following random integration of a vector carrying this GS variant (but with reduced cell growth, indicating reduced efficiency of the enzyme. Data not shown). To evaluate if other amino acids/codons at position 299 can yield Glutamine Synthetase enzymes with higher efficiency compared to the negative control, a met mutant and a library containing 29 functional codons representing 18 different amino acids were tested in a Site Directed Integration workflow using HyClone CHO LP2P2.

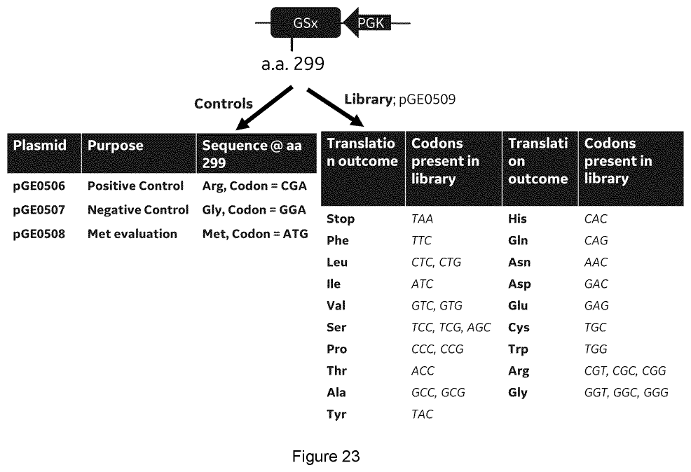

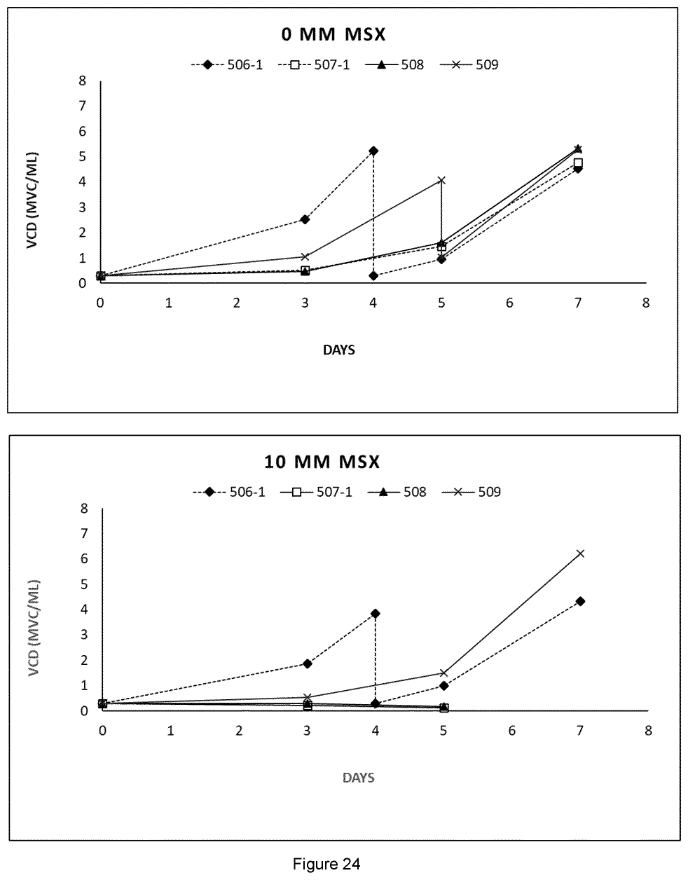

HyClone CHO LP2P2 were transfected with each of the three control plasmids and the plasmid library in separate transfections. Cell pools selected for integration of a single DNA Donor Vector copy at the Landing Pad were generated according to the workflow described in Example 4. As an initial test of GS functionality and as an enrichment step of functional variants in the library, each final pool of cells were cultured in media lacking L-Glutamine either in the absence of MSX or in the presence of 10 μM MSX. Growth curves can be seen in FIG. 24. As can be seen all variants can grow in the absence of MSX but with varying growth rates. The positive control grows fastest, followed by the library. The negative control and the met mutant had the slowest growth rate. In the presence of 10 μM MSX only the positive control and the library grow, with the positive control growing at a higher rate.

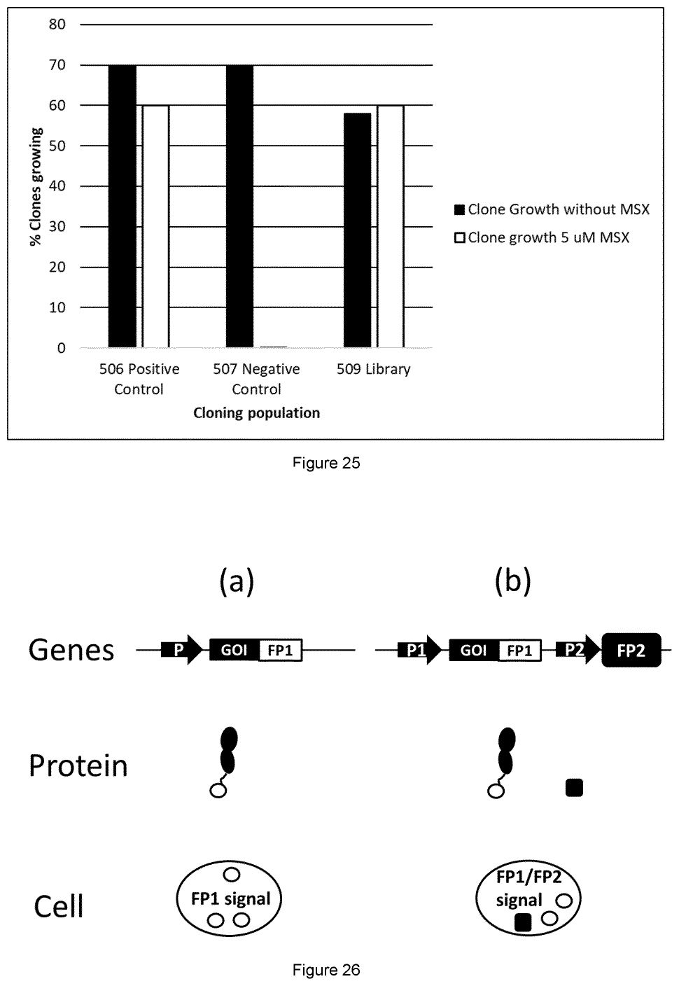

Following growth with 10 μM MSX as selection for functional GS activity, single cells were cloned using FACS into 96-well static culture plates from the library culture, the positive control culture and the negative control culture. Single cells were grown in glutamine free media in the absence of MSX or in the presence of 5 μM MSX. Wells were monitored for growth using a Solentim plate imaging system. A graph illustrating the outgrowth for the library as compared with the controls can be found in FIG. 25.

As can be seen, clones grow out for all variants in the absence of MSX. For culture using 5 μM MSX, clones do not grow out for the negative control but do grow for the positive control and clones from the library.

To identify GS variants showing outgrowth in the presence of 5 μM MSX, genomic DNA were extracted from corresponding wells and the GS region amplified by PCR. A total of 96 PCR products from 96 clones were sent for sanger sequencing and high-quality sequencing results were obtained for 79 clones. Sequences from the sanger sequencing were aligned to GS variants using the Geneious Prime software. A summary of the GS variants identified can be found in Table 5.

TABLE 5
Summary of GS variants from clones growing out in the presence
of 5 μM MSX and identified by Sanger sequencing.

| Amino | Clone | |
|---|---|---|
| Acid | frequency | Codon frequency |
| Arg | 99% (78/79) | ACG - 56% (44/78) |
| | | CGG - 30% (24/78) |
| | | CGC - 13% (10/78) |
| Gly | 1% (1/79) | GGG (1/1) |

As can be seen, 99% of the identified clones have Arg at pos 299 and only one variant has Gly. The fact that 99% of the variants identified correspond to the same amino acid as in the positive control (but with other codons) illustrates the utility and precision of the SDI system for evaluating libraries of sequence variants in a parallel workflow. It should also be noted that the three different Arg codons present in the library are all identified from the outgrown clones but with clearly different frequencies, indicating that the effect of a single synonymous codon change can be picked up by the methodology.

### REFERENCES

[1] Ecker, D M, et al; The therapeutic monoclonal antibody market; mAbs 7:1, 9-14; Jan./Feb. 2015.
[2 ] Kunert, R, et. al.; Advances in recombinant antibody manufacturing; Appl Microbiol Biotechnol (2016) 100:3451-3461.
[3 ] Labrijn, A F, et al.; Bispecific antibodies: a mechanistic review of the pipeline; NATURE Reviews| Drug Discovery volume 18| AUGUST 2019.
[4 ] Wang, Q, et al.; Design and Production of Bispecific Antibodies; Antibodies 2019, 8, 43; doi:10.3390/antib8030043.
[5 ] Meinke, G, et al; Cre Recombinase and Other Tyrosine Recombinases; Chem. Rev. 2016, 116, 12785-12820.
[6 ] Merrick, C A; et al.; Serine Integrases: Advancing Synthetic Biology; ACS Synth. Biol. 2018, 7, 299-310.
[7 ] Xu, Z, et al.; Accuracy and efficiency define Bxb1 integrase as the best of fifteen candidate serine recombinases for the integration of DNA into the human genome; BMC Biotechnology 2013, 13:87.
[8 ] Lee, J S, et al.; Accelerated Homology-Directed Targeted Integration of Transgenes in Chinese Hamster Ovary Cells Via CRISPR/Cas9 and Fluorescent Enrichment; Biotechnol. Bioeng. 2016; 9999: 1-6.
[9 ] Invitrogen; Flp-In system for generating stable mammalian expression cell lines by Flp recombinase-mediated integration; Invitrogen Instruction Manual 2001; Invitrogen, Carlsbad CA.
[10] Muller, D; Accelerating Time to Clinical Manufacturing Following a Targeted Gene Integration Approach; Bioprocess International Conference, Boston; Oct. 28, 2015.
[11 ] Haghighat-Khah, R E, et al.; Site-Specific Cassette Exchange Systems in the Aedes aegypti Mosquito and the Plutella xylostella Moth; PLOS ONE| DOI:10.1371/journal.pone.0121097 Apr. 1, 2015.
[12 ] Yuan, Y, et al.; Improved site-specific recombinase-based method to produce selectable marker- and vector-backbone-free transgenic cells; SCIENTIFIC REPORTS| 4: 4240| DOI: 10.1038/srep04240.
[13 ] Parthiban, K, et al.; A comprehensive search of functional sequence space using large mammalian display libraries created by gene editing; MABS 2019, VOL. 11, NO. 5, 884-898.
[14 ] Gupta, K, et al.; Vector-related stratagems for enhanced monoclonal antibody production in mammalian cells; Biotechnology Advances 37 (2019) 107415.
[15 ] Noderer, W L, et al.; Quantitative analysis of mammalian translation initiation sites by FACS-seq; Molecular Systems Biology 10: 748| 2014.
[16 ] Stern, B, et al.; Improving mammalian cell factories: The selection of signal peptide has a major impact on recombinant protein synthesis and secretion in mammalian cells; T r e n d s in Cell & Molecular B i o l o g y, 2007.
[17 ] Haryadi, R, et al.; Optimization of Heavy Chain and Light Chain Signal Peptides for High Level Expression of Therapeutic Antibodies in CHO Cells; PLOS ONE| DOI:10.1371/journal.pone. 0116878 Feb. 23, 2015.
[18 ] Pearson, M J, et al.; Albumin 3′ untranslated region facilitates increased recombinant protein production from Chinese hamster ovary cells; Biotechnol. J. 2012, 7, 1405-1411.
[19 ] Hanson, G, et al.; Codon optimality, bias and usage in translation and mRNA decay; Nat Rev Mol Cell Biol. 2018 January; 19(1): 20-30.
[20 ] Shendure, J, et al.; DNA sequencing at 40: past, present and future; Nature volume 550, pages 345-353(2017).
[21] Periyannan Rajeswari, P K, et al, Droplet size influences division of mammalian cell factories in droplet microfluidic cultivation; Electrophoresis 2017, 38, 305-310.
[22 ] King, D, et al.; Single Cell Analysis Microfluidic Device for Cell Line Optimisation in Upstream Cell Culture Processing Biopharamceutical Applications; 2019 20th International Conference on Solid-State Sensors, Actuators and Microsystems & Eurosensors XXXIII (TRANSDUCERS & EUROSENSORS XXXIII).
[23] Le, K, et al.; A Novel Mammalian Cell Line Development Platform Utilizing Nanofluidics and OptoElectro Positioning Technology; Biotechnol. Prog., 2018, Vol. 34, No. 6.
[24 ] EP 2 329 020 B1; CELL SURFACE DISPLAY OF POLYPEPTIDE ISOFORMS BY STOP CODON READTHROUGH.
[25] Fink, T, et al.; Design of fast proteolysis-based signaling and logic circuits in mammalian cells; Nat ure Chemical Biology| VOL 15| FEBRUARY 2019| 115-122|.
[26 ] Kuo, C C, et al.; The emerging role of systems biology for engineering protein production in CHO cells; Current Opinion in Biotechnology Volume 51, June 2018, Pages 64-69.
[27 ] Chiu, M L, et al.; Antibody Structure and Function: The Basis for Engineering Therapeutics; Antibodies 2019, 8, 55; doi:10.3390/antib8040055.
[28 ] Saerens, D, et al.; Single-domain antibodies as building blocks for novel therapeutics; Current Opinion in Pharmacology 2008, 8:600-608.
[29 ] Gebauer, M, et al.; Engineered Protein Scaffolds as Next-Generation Therapeutics; Annu. Rev. Pharmacol. Toxicol. 2020. 60:391-415.
[30 ] Zhou, C, et al.; Development of a novel mammalian cell surface antibody display platform; mAbs 2:5, 508-518; September/October 2010.
[31 ] Ferreira, J P, et al.; Tuning gene expression with synthetic upstream open reading frames; PNAS| Jul. 9, 2013| vol. 110| no. 28.

1. A method for the selection of a sequence optimized nucleic acid sequence from a plurality of nucleic acid sequence variants, wherein said sequence optimized nucleic acid sequence corresponds to a eukaryotic cell with a defined phenotype, said method comprising:
i) Providing a population of isolated eukaryotic cells, each cell comprising a pre-defined genomic location, which pre-defined genomic location comprises:
a. a nucleic acid sequence I1 comprising a recognition site for a first DNA enzyme;
b. a nucleic acid sequence E1 comprising a recognition site for a second DNA enzyme; and
c. a promotor nucleic acid sequence;
ii) Providing a plurality of donor vectors, each donor vector comprising:
a. a nucleic acid sequence I2;
b. a nucleic acid sequence E2 comprising a recognition site for said second DNA enzyme;
c. a nucleic acid sequence encoding a first selection marker; and
d. a nucleic acid sequence region comprising a nucleic acid sequence variant,
iii) Contacting the plurality of donor vectors with the population of cells in the presence of a first DNA enzyme, wherein the presence of the first DNA enzyme enables recombination between the nucleic acid sequence I2 of a donor vector and the nucleic acid sequence I1 present in the pre-defined genomic location of a cell;
iv) Selecting and isolating a cell having a donor vector integrated at the pre-defined genomic location by detecting the expression of the first selection marker in the cell, wherein the expression of the first selection marker is activated by the promotor nucleic acid sequence at the pre-defined genomic location; and
v) Selecting and isolating a cell with a defined phenotype from the cells of step iv), thereby selecting and isolating a sequence optimized nucleic acid sequence from said nucleic acid sequence variants, said sequence optimized nucleic acid sequence corresponding to said defined phenotype.

2. The method of claim 1, wherein each of the plurality of donor vectors of step ii) further comprises:
e. an expression cassette encoding a second selection marker; and
wherein said method further comprises the steps of:
vi) Excising a nucleic acid sequence flanked by the nucleic acid sequences E1 and E2 from a cell of step iii) or selected and isolated in step iv) or step v) in the presence of a second DNA enzyme, wherein the presence of the second DNA enzyme enables recombination between the nucleic acid sequences E1 and E2, wherein the presence of an expression cassette encoding a second selection marker that is not flanked by the nucleic acid sequences E1 and E2 in a cell is indicative of a stable integration of a donor vector at a genomic location outside the pre-defined genomic location of said cell; and
vii) Selecting and isolating a cell from step vi) lacking an expression cassette encoding a second selection marker.

3. The method of claim 1, further comprising obtaining nucleic acid sequence information from the isolated cell of step iv), v) or vii) by sequencing of the sequence optimized nucleic acid sequence present at the pre-defined genomic location of the isolated cell.

4. The method of claim 1, wherein a nucleic acid sequence variant of the plurality of donor vectors constitutes a variant of a promotor, a variant of an intron, a variant of a transcription regulatory sequence, a variant of a DNA structure regulatory sequence, a variant of a 5′ untranslated region, a variant of a 3′ untranslated region, a variant of an internal ribosome entry site, a variant of a gene of interest, a variant of a nucleic acid sequence encoding a signal peptide and/or any combination of such variant.

5. The method of claim 1, wherein the selection of a sequence optimized nucleic acid sequence by the selection of a cell with a defined phenotype in step v) of claim 1 comprises selection based on one or more of the following phenotypic characteristics of said cell:
(i) The presence or expression level of an endogenous biomolecule in said cell;
(ii) The expression level of a recombinant protein of interest by said cell;
(iii) The growth rate of said cell; and/or
(iv) The functionality of a recombinant protein of interest encoded by said nucleic acid sequence region in said cell.

6. The method of claim 5, wherein said recombinant protein of interest is a recombinant fusion protein, such as a protein fused to a membrane anchor domain for localization at said cell surface and/or is a protein fused to a fluorescent protein or a fluorescent protein domain.

7. The method of claim 5, wherein said endogenous biomolecule constitutes a protein, an mRNA, an miRNA, an lncRNA or a metabolite.

8. The method of claim 5, wherein said functionality of a recombinant protein of interest is measured and determined based on an interaction between said recombinant protein of interest when localized and expressed at the cell surface of said cell, and a target structure, such as a small molecule, a DNA molecule, an RNA molecule, a protein, a protein complex such as a virus particle, an exosome or a cell, optionally wherein said target structure is tagged with a fluorescent moiety.

9. The method of claim 5, wherein said recombinant protein of interest is an affinity protein candidate and wherein the level of expression is determined by display of said affinity protein candidate on the cell surface of said cell.

10. The method of claim 9, wherein said affinity protein candidate is a single chain polypeptide fused to a membrane anchor domain, optionally wherein said single chain polypeptide is selected from the group consisting of a Z-scaffold protein, a Nanobody scaffold protein, a single chain fragment variable (scfv) scaffold protein, a Fynomer scaffold protein, a DARPin scaffold protein and/or an adnectin scaffold protein.

11. The method of claim 9, wherein said affinity protein candidate comprises two or more polypeptide chains, such as an antibody variant, and wherein a nucleic acid sequence variant corresponding to said affinity protein candidate encodes an affinity protein candidate variant, such as an antibody variant, and wherein one of said two or more polypeptides is fused to a membrane anchor domain.

12. The method of claim 9, further comprising determining the binding specificity, selectivity, affinity and/or functionality of said affinity protein candidate by providing to said affinity protein candidate a specific target component, optionally labelled with a fluorescent marker, to which the affinity protein candidate is exposed and thereafter detecting binding of said affinity protein candidate to said specific target component.

13. The method of claim 12, wherein said target component is selected from a small molecule, a DNA molecule, an RNA molecule, a protein, a protein complex, such as a virus particle, an exosome or a cell, optionally wherein said target structure is tagged with a fluorescent moiety.

14. The method of claim 5, wherein said expression level or functionality of a recombinant protein of interest and/or said presence or level of an endogenous biomolecule is measured at the level of a single cell, such as by using Flow Cytometry.

15. The method of claim 1, wherein said nucleic acid sequence region of a donor vector comprises a nucleic acid sequence variant for expression of one or several recombinant proteins of interest from said donor vector, wherein said plurality of donor vectors comprises different nucleic acid sequence variants encoding different amino acid sequence variants of said one or several recombinant proteins of interest.

16. The method of claim 1, wherein said nucleic acid sequence region of a donor vector comprises a nucleic acid sequence variant for expression of a recombinant protein of interest from said donor vector, wherein nucleic acid sequence variants present in a plurality of donor vectors comprises nucleic acid sequence variants encoding essentially identical amino acid sequence variants of said recombinant protein of interest.

17. The method of claim 1, comprising a plurality of donor vectors comprising a nucleic acid sequence region comprising essentially identical nucleic acid sequences to encode essentially identical recombinant proteins of interest but wherein said nucleic acid sequence region comprises different nucleic acid sequence variants of donor vector components, such as nucleic acid sequence variants of promoter or enhancer nucleic acid sequences of said donor vector.

18. The method of claim 1, wherein said method identifies sequence optimized nucleic acid donor vector components for use in recombinant protein expression systems based on a eukaryotic cell system.

19. A sequence optimized nucleic acid sequence selected by a method of claim 1.

20. Use of a sequence optimized nucleic acid sequence of claim 19, for producing a recombinant protein.

21. Use of a sequence optimized nucleic acid sequence of claim 19, for designing further sequence optimized nucleic acid sequences.

22. An isolated eukaryotic cell with a defined phenotype corresponding to a sequence optimized nucleic acid sequence obtainable by the method of claim 1.


SEQUENCE LISTING (16 sequences)

SEQ ID NO: 1 - DNA, 47 bp, Artificial sequence
  Partly modified native attP sequence
gtgccccaactggggtaacctttgagttctctcagttgggggcgtag

SEQ ID NO: 2 - DNA, 70 bp, Artificial sequence
  Partly modified native attB sequence
ctcgaagccgcggtgcgggtgccagggcgtgcccttgggctccccgggcgcgtactccacctcacccatc

SEQ ID NO: 3 - DNA, 47 bp, Artificial sequence
  Partly modified native attP sequence
gtgccccaactggggtaacctaagagttctctcagttgggggcgtag

SEQ ID NO: 4 - DNA, 70 bp, Artificial sequence
  Partly modified native attB sequence
ctcgaagccgcggtgcgggtgccagggcgtgcccaagggctccccgggcgcgtactccacctcacccatc

SEQ ID NO: 5 - DNA, 1941 bp, Artificial sequence
  Partly modified native PhiC31 gene
atgaccatgattaccccatctgcccagctgaccctgacaaagggcaataagagctggtctagcctggtgacagctgcttctgtgctggagtttgccaccatgatccaaggggtcgctggggaagtgacttatgccggggcgtacgaccgtcagtctcgggagcgcgagaactctagcgcggcgtctccggccactcagcgtagcgctaacgaggccaaagccgccgctctccagcgcgagatcgagcgcgccgggggccggtttcgtttcgtcggtcacttcagcgaggcccccggcacatctgccttcggtacagccgagcgccctgagttcgaacgcattctgaacgaatgccgcgccggtcggctgaacatgattatcgtgtatgacgtgtctcgcttctctcgcctgaaggttatggacgccatccctatcgtgtcagaattactggccctgggcgtgacaatcgtctctacgcaggaaggcgtgttcagacaagggaacgttatggacctgatccacctgatcatgcggctggacgcctctcacaaagaaagctctctgaagtctgccaagatcctggacacaaagaacctccagcgcgaacttggcggttacgtgggcgggaaggccccctacggcttcgagcttgtcagcgagacaaaggagattacacgcaacggacgtatggtcaatgtggttatcaacaagctcgcccactctaccacgcctctcaccggacctttcgagttcgagccagacgtaattcggtggtggtggcgtgagatcaagacacacaaacacctccctttcaagcctggcagtcaagccgccatccaccctggctctattaccggactctgtaagcgcatggacgcggacgccgtgcctaccagaggcgagacaatcgggaagaagaccgcgtcgtctgcctgggaccctgcgaccgtcatgcgtattctcagagaccctcgtatcgccgggttcgctgcggaggtgatttacaagaagaagccagacggcacacctaccacaaagatcgagggataccgcatccagcgcgaccctattactctgcggcctgtggagcttgattgcggtcctattatcgagcctgcggagtggtatgagcttcaggcctggttggacggacgtggtcgcggcaagggtctctctcggggtcaagccatcctgtctgctatggacaagctgtactgcgagtgtggcgccgttatgacgagcaagcgcggggaagaatctatcaaggacagttaccgctgccgtcgcagaaaggtggtggacccttctgcgcccggtcagcacgaaggcacttgcaacgtctctatggccgcgctggacaagttcgtcgccgaacgcattttcaacaagatccgtcacgccgaaggcgacgaagagacacttgccctcctgtgggaagccgcccgtcgcttcggcaagctcacggaggcccccgagaagtctggcgaaagagccaacctcgtcgccgagcgcgccgacgccctgaacgccctcgaagagctgtacgaagaccgcgctgcgggcgcctacgacggtcctgtcggacgaaagcacttcagaaagcaacaggcggccctgactctgcgccagcaaggtgccgaagagagactcgccgaactcgaagccgccgaagccccaaagctccctctcgaccaatggttcccagaagacgccgacgcggaccctaccggccccaagtcttggtggggtcgcgcctcggtagacgacaagcgcgtgttcgtgggtctgttcgtagacaagattgtcgttacaaagtctacgacaggccgtgggcaggggacacctatcgagaagcgcgcgtctattacttgggccaagcctcctaccgacgacgacgaagacgacgcccaggacggcacagaagacgtagctgcttgataa

SEQ ID NO: 6 - DNA, 34 bp, Artificial sequence
  Partly modified native lox gene
ataacttcgtataggatactttatacgaagttat

SEQ ID NO: 7 - DNA, 1031 bp, Artificial sequence
  Partly modified native Cre gene
atgtcaaaccttctcaccgtccaccaaaacctccccgcactccccgttgacgccacctccgacgaggtcagaaaaaacctcatggacatgttccgggaccgccaggccttttccgaacacacttggaaaatgcttctcagcgtttgccgtagttgggccgcttggtgtaaactcaacaaccgcaagtggttccccgccgaacccgaggacgtccgcgattaccttctgtatttgcaagcgcgaggactggccgtgaaaaccatccagcaacatctgggtcagcttaacatgttgcaccggaggagcggcctgccacggcctagcgactccaacgcggtgtccctcgtgatgaggagaatccgcaaggagaatgtggacgccggagaaagagcaaagcaggccctggccttcgagaggactgacttcgaccaagtccggtcgctgatggagaactcggaccgatgtcaggacatcaggaacctcgcattctcggcattgcctacaacaccctgctgagaattgcagagatcgcccgcatccgcgtcaaggacatttcgagaaccgacggagggcggatgctgattcacatcggcaggactaagaccctcgtgtcaaccgccggagtggaaaaggccctcagcctgggagtgacaaagctcgtggagcgctggatctccgtgtcgggggtggccgacgatccgaacaattacctgttctgccgggtccgcaaaaatggggtggccgccccgtctgctacaagccagttgtccactcgcgccctggaaggaatcttcgaggccacgcaccgcctgatctatggggcaaaggacgattccggccagaggtatctcgcgtggtccggtcactccgcgcgcgtgggcgcggcccgggacatggcccgggctggagtgtccatccctgaaatcatgcaggccggtggatggaccaacgtgaacatcgtgatgaactacattcggaacctggacagcgaaactggtgctatggtccgcctgctggaggacggagattga

SEQ ID NO: 8 - DNA, 1428 bp, Artificial sequence
  Enhanced Green Fluorescent Protein fused to an FC from IgG1 (FC-eGFP gene)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggttcctccagttccggcagctccagttccggtatgagtaaaggagaggaactcttcaccggagtcgtcccgatactcgtcgagctagacggagacgtcaacggccacaaattctccgtctccggcgagggggagggggacgccacctacggaaaactcacccttaagtttatttgcactaccggaaaactccccgtcccttggccaaccctagtcaccacgctgacatacggagtccaatgtttctcgcggtatcccgaccacatgaagcagcatgactttttcaaatccgcgatgcctgagggctacgtgcaggaacgcaccatcttcttcaaggacgacgggaattacaagactagagccgaggtcaagtttgaaggagacaccctcgtgaatcgcatcgagcttaagggcattgacttcaaggaggacggcaacatcctgggtcacaagctggagtacaactacaactcgcataacgtctacatcatggccgacaagcaaaagaacggtatcaaggtcaacttcaagattaggcacaacattgaggatgggtccgtccaactggccgaccactaccagcagaacacccccatcggcgacggacctgtgctcctgcctgataaccactatctcagcactcagagcgcactgtccaaggaccctaacgaaaaacgggaccacatggtcttgctggagttcgtgacagccgctggtattaccctgggcatggatgaactgtataag

SEQ ID NO: 9 - DNA, 1431 bp, Artificial sequence
  Blue Fluorescent Protein variant fused to an FC from IgG1 (FC-TagBFP2 gene)
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccacgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggttcctccagttccggcagctccagttccggtatggtgtcgaagggagaggagctgattaaggagaacatgcacatgaagctgtatatggaagggacggtggacaaccaccacttcaagtgcaccagcgaaggagaaggaaagccttacgaaggcactcaaactatgcggatcaaagtggtggaaggcggtcctcttccgttcgccttcgacatcttggccacctccttcctctacggctccaagacctttatcaaccacacccagggaatcccggacttctttaagcagagcttccctgagggcttcacctgggaaagagtgacaacctacgaggacggtggcgtcctgaccgcgacccaggacacctccctgcaagacggctgcctgatctacaacgtcaagattcgcggcgtgaacttcacctccaatggtccagtgatgcagaagaaaactctgggatgggaggccttcactgaaactctgtaccccgccgatggaggactggaggggaggaacgatatggctttgaagctcgtggggggatcgcacctgattgcgaatgccaagaccacctacagatccaagaaacccgccaagaacctcaagatgcccggagtctactacgtggactatagactggaacggatcaaggaagccaacaacgagacttacgtggaacagcacgaggtcgctgtggcacgctactgtgatctgccgtcaaagctcgggcataagctcaactgataa

SEQ ID NO: 10 - DNA, 738 bp, Artificial sequence
  Red Fluorescent Protein Variant (TagRFP-T gene)
atggtgtcaaagggagaggaactgattaaggagaatatgcacatgaaactctacatggaggggaccgtgaacaaccaccacttcaagtgcacctccgagggcgaagggaagccgtacgagggaactcagaccatgcggattaaggtcgtcgaagggggtcctctgccattcgccttcgacatcctcgccacatcctttatgtacggatcgcggaccttcatcaaccacactcagggtatccccgacttcttcaagcaatcgttcccggaaggctttacttgggagcgcgtgaccacctacgaggatggaggggtgctgacggccactcaggacaccagcctgcaagacggctgtcttatctacaacgtgaagattcgcggcgtgaacttccctagcaacggtccggtcatgcagaaaaagaccctgggttgggaggctaacaccgaaatgctctatcctgcggacggaggattggaaggccggactgacatggccctgaaacttgtgggcggcggacatctgatctgcaatttcaagaccacttaccgctccaagaagcccgccaagaacctgaagatgcctggagtgtactacgtggaccacagactcgaaaggatcaaggaggcggataaggaaacctacgtggaacagcatgaagtggcagtggccagatactgcgatctgccgtccaagctcggccacaagctgaacggaatggacgagctgtataagtgataa

SEQ ID NO: 11 - DNA, 714 bp, Artificial sequence
  Enhanced Green Fluorescent Protein (eGFP gene)
atgagtaaaggagaggaactcttcaccggagtcgtcccgatactcgtcgagctagacggagacgtcaacggccacaaattctccgtctccggcgagggggagggggacgccacctacggaaaactcacccttaagtttatttgcactaccggaaaactccccgtcccttggccaaccctagtcaccacgctgacatacggagtccaatgtttctcgcggtatcccgaccacatgaagcagcatgactttttcaaatccgcgatgcctgagggctacgtgcaggaacgcaccatcttcttcaaggacgacgggaattacaagactagagccgaggtcaagtttgaaggagacaccctcgtgaatcgcatcgagcttaagggcattgacttcaaggaggacggcaacatcctgggtcacaagctggagtacaactacaactcgcataacgtctacatcatggccgacaagcaaaagaacggtatcaaggtcaacttcaagattaggcacaacattgaggatgggtccgtccaactggccgaccactaccagcagaacacccccatcggcgacggacctgtgctcctgcctgataaccactatctcagcactcagagcgcactgtccaaggaccctaacgaaaaacgggaccacatggtcttgctggagttcgtgacagccgctggtattaccctgggcatggatgaactgtataag

SEQ ID NO: 12 - DNA, 717 bp, Artificial sequence
  Blue Fluorescent Protein variant (TagBFP2 gene)
atggtgtcgaagggagaggagctgattaaggagaacatgcacatgaagctgtatatggaagggacggtggacaaccaccacttcaagtgcaccagcgaaggagaaggaaagccttacgaaggcactcaaactatgcggatcaaagtggtggaaggcggtcctcttccgttcgccttcgacatcttggccacctccttcctctacggctccaagacctttatcaaccacacccagggaatcccggacttctttaagcagagcttccctgagggcttcacctgggaaagagtgacaacctacgaggacggtggcgtcctgaccgcgacccaggacacctccctgcaagacggctgcctgatctacaacgtcaagattcgcggcgtgaacttcacctccaatggtccagtgatgcagaagaaaactctgggatgggaggccttcactgaaactctgtaccccgccgatggaggactggaggggaggaacgatatggctttgaagctcgtggggggatcgcacctgattgcgaatgccaagaccacctacagatccaagaaacccgccaagaacctcaagatgcccggagtctactacgtggactatagactggaacggatcaaggaagccaacaacgagacttacgtggaacagcacgaggtcgctgtggcacgctactgtgatctgccgtcaaagctcgggcataagctcaactgataa

SEQ ID NO: 13 - DNA, 792 bp, Artificial sequence
  Neomycin resistance gene (NeoR gene)
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 14 - DNA, 1122 bp, Artificial sequence
  Partially modified native Glutamine Synthetase gene (GS gene)
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccgctaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaactgtgacccctttgcagtgacagaagccatcgtccgcacatgccttctcaatgagactggcgacgagcccttccaatacaaaaactaa

SEQ ID NO: 15 - DNA, 39 bp, Artificial sequence
  462 5′ part of 5′UTR
accatgggttgaaccatgggttgaaccatgggttgaacc

SEQ ID NO: 16 - DNA, 15 bp, Artificial sequence
  464 5′ part of 5′UTR
caaatgggttgaacc